(19) United States
(12) Patent Application Publication
(10) Pub. No.: US 2024/0358167 A1
(43) Pub. Date: Oct. 31, 2024

(54) SMART MATTRESS WITH ADAPTIVE ACTUATION SYSTEM

(71) Applicant: PROHIBITION X PTE LTD, Singapore (SG)

(72) Inventor: Stephen D. Newman, Singapore (SG)

(73) Assignee: PROHIBITION X PTE LTD, Singapore (SG)

(21) Appl. No.: 18/649,337

(22) Filed: Apr. 29, 2024

Related U.S. Application Data
(60) Provisional application No. 63/499,147, filed on Apr. 28, 2023.

(51) Int. Cl. A47C 27/08; A47C 27/10; A47C 31/12; A61M 21/00; A61M 21/02

(52) U.S. Cl. CPC A47C 27/083; A47C 27/10; A47C 31/123; A61M 21/02; A61M 2021/0016; A61M 2021/0022; A61M 2021/0027; A61M 2021/0044; A61M 2230/10

(57) ABSTRACT

A smart mattress and adaptive actuation systems for a flexible fabric are disclosed. The mattress can include a flexible fabric associated with a sleeping or resting structure configured to engage a user arranged in a prone, semi-prone, or sitting position. An actuation assembly can be configured to manipulate the sleeping or resting structure. In one example, the actuation assembly includes a plurality of size-changing components, and the sleeping or resting structure can be responsive to sizes of the size-changing components. The actuation assemblies can be included in a sealable sleeping or resting structure, which can alter at least a vibration, a light, a sound, or a composition and pressure of a gaseous environment of a user.

### CROSS-REFERENCE TO RELATED APPLICATIONS

This application claims priority to and the benefit of U.S. Provisional Patent Application No. 63/499,147 filed Apr. 28, 2023 entitled “SMART MATTRESS WITH ADAPTIVE ACTUATION SYSTEM,” the disclosure of which is incorporated herein in its entirety by reference.

### FIELD

The described embodiments relate generally to actuation systems for fabric, and more particularly, to systems and techniques for adaptively controlling characteristics of a smart mattress or other cushioning device.

### BACKGROUND

Conventional smart mattresses utilize automatic air-filled bladders or cells to adjust the firmness of a mattress to a user preference. Air-filled bladders are often poorly suited to support a user and/or to adapt to movements of the user. Rather than help maintain spinal support, the conventional air-filled bladders can be easily deformed and create a sensation of displacing air, which can lead to the user being unintentionally rolled. Conventional smart mattresses can also suffer from a variety of drawbacks that result from an overall complex design and reliance on high-power-consumption motors to adjust air pressure in an individual cell. Leaks in the bladder, power failures, wear, and so on can cause deflation of conventional systems and contribute to generally poorer reliability that detracts from the user experience.

### SUMMARY

In one aspect, a resting or sleeping system includes a sleeping or resting structure, an actuation assembly, and a chamber. The sleeping or resting structure can be configured to engage a user arranged in a sitting, prone, or semi-prone position. The actuation assembly can be configured to manipulate the sleeping or resting structure. The sleeping or resting structure can be responsive to energy provided by the actuation assembly. The chamber can be configured to manipulate a sleeping or resting environment of the user via control of at least one of a vibration, a light, a sound, or a gas.

In some examples, the resting or sleeping system further includes a sensing module that can be configured to detect brainwaves of the user. In some examples, the chamber can be configured to manipulate the sleeping or resting environment in response to the brainwaves detected by the sensing module.

In some examples, the sleeping chamber can be configured to provide gaseous medical treatments to the user.

In some examples, the actuation assembly can include a size-changing component. In some examples, the size-changing component can be configured to change size in response to energy supplied to the size-changing component. In some examples, the sleeping or resting structure can be responsive to the size of the size-changing component. In some examples, the size-changing component can include a material having a volumetric coefficient of thermal expansion greater than 20/° C. In some examples, the size-changing component can be spherical.

In some examples, the size-changing component has a free end; the free end is in a first position when the size-changing component has a first size; and the free end is in a second position when the size-changing component has a second size.

In one aspect, a method for providing an immersive sleep or rest experience includes providing a flexible fabric in a sleeping volume, causing a deformation of the flexible fabric, and altering at least one of a composition of gas or a pressure of gas in the sleeping volume. The flexible fabric can be configured to support a user in the sleeping volume. The deformation of the flexible fabric can be caused by manipulating an actuator.

In some examples, causing the deformation of the flexible fabric can include transitioning a size-changing component from a first size to a second size. In some examples, the size-changing component can be transitioned from the first size to the second size by altering energy supplied to the size-changing component.

In some examples, the method can further include detecting a user input. In some examples, the user input can include data from a user's smart device. In some examples, transitioning the size-changing component can occur in response to the detection of the user input.

In some examples, the data can include one or more of daily activities, oxygen levels, heart rate, diet, consumables, or environmental factors.

In some examples, the method can further include detecting a user input. In some examples, the user input can include data from a user's smart device. In some examples, altering at least one of the composition of gas or the pressure of gas in the sleeping volume can occur in response to the detection of the user input.

In some examples, the method can further include detecting the user's brainwaves. In some examples, altering at least one of the composition of gas or the pressure of gas in the sleeping volume can occur in response to the detection of the user's brainwaves.

In some examples, the method can further include altering thermal energy supplied to a fluid in a temperature control system to alter a temperature of the flexible fabric.

In some examples, the method can further include supplying a gaseous medical treatment to the sleeping volume.

In one aspect, a mattress can include a sleeping or resting structure and an actuation assembly. The sleeping or resting structure can be configured to engage a user arranged in a sitting, prone, or semi-prone position. The actuation assembly can be configured to manipulate the sleeping or resting structure. The actuation assembly can include a size-changing component configured to transition between a first size and a second size in response to the size-changing component receiving energy from an energy source. The sleeping or resting structure can be responsive to the size-changing component having the first size or the second size

In some examples, the size-changing component can include a material having a volumetric coefficient of thermal expansion greater than 20/° C.

In some examples, the energy source can include a heat source; the heat source can be configured to emit heat directed toward the size-changing component; and the size-changing component can be configured to transition from the first size to the second size upon the receipt of heat from the heat source.

In some examples, the actuation assembly can include a plurality of size-changing components including the size-changing components. In some examples, the size-changing components can be spherical.

In some examples, the size-changing component can include a plurality of shapeable materials. In some examples, the shapeable materials can be configured to transition from a first shape to a second shape upon the receipt of energy to transition the size-changing component from the first size to the second size.

In addition to the exemplary aspects and embodiments described above, further aspects and embodiments will become apparent by reference to the drawings and by study of the following description.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 depicts an example mattress;

FIG. 2 depicts a functional diagram of an adaptive actuation system;

FIG. 3A depicts an example mattress with the adaptive actuation system of FIG. 2 and having a user arranged in a first lying position;

FIG. 3B depicts the mattress of FIG. 3A with the user arranged in a second lying position;

FIG. 4A depicts a flexible fabric with an actuation assembly in a first configuration;

FIG. 4B depicts the flexible fabric of FIG. 4A with the actuation system in a second configuration;

FIG. 4C depicts the flexible fabric of FIG. 4A with another example actuation system;

FIG. 5A depicts another flexible fabric with an actuation system in a first configuration;

FIG. 5B depicts the flexible fabric of FIG. 5A with the actuation system in a second configuration;

FIG. 5C depicts the flexible fabric of FIG. 5A with another example actuation system;

FIG. 6 depicts another example flexible fabric having an array of shapeable materials;

FIG. 7A depicts an example shapeable material in a first configuration;

FIG. 7B depicts the shapeable material of FIG. 7B in a second configuration;

FIG. 8 depicts a schematic diagram of a photopolymer or light activated resin;

FIG. 9A depicts another example shapeable material in a first configuration;

FIG. 9B depicts the shapeable material of FIG. 9A in a second configuration;

FIG. 9C depicts the shapeable materials of FIGS. 9A and 9B arranged for supporting a mattress;

FIG. 10A depicts an example wavefront sensor measuring a first wavefront;

FIG. 10B depicts the example wavefront sensor measuring a second wavefront;

FIG. 11A depicts a sleep system with an adaptive actuation system and having a user arranged in a first lying position;

FIG. 11B depicts the sleep system of FIG. 11A having the user arranged in a first lying position;

FIG. 11C depicts a schematic view of the sleep system of FIG. 11A;

FIG. 12A depicts a first user interface of an electronic device configured to operate the sleep system of FIG. 11A;

FIG. 12B depicts a second user interface of an electronic device configured to operate the sleep system of FIG. 11B;

FIG. 12C depicts a first user interface of an electronic device configured to operate the sleep system of FIG. 11C;

FIG. 13A depicts a medical table with an adaptive actuation system and having a user arranged in a first convalescing position;

FIG. 13B depicts the medical table of FIG. 13A having the user arranged in a second convalescing position;

FIG. 14A depicts a surgical table with an adaptive actuation system and having a user arranged in a first surgical position;

FIG. 14B depicts the surgical table of FIG. 14A having the user arranged in a second surgical position;

FIG. 15A depicts a car seat with an adaptive actuation system and having a user arranged in a first seated position;

FIG. 15B depicts the car seat of FIG. 15A having the user arranged in a second seated position;

FIG. 16A depicts a garment with an adaptive actuation system and having a user arranged in a first position;

FIG. 16B depicts the garment of FIG. 16A having the user arranged in a second position;

FIG. 17A depicts an insole with an adaptive actuation system and having a foot of a user arranged in a first position;

FIG. 17B depicts the insole of FIG. 17A having the foot of the user arranged in a second position;

FIG. 18A depicts a bulletproof vest with an adaptive actuation system and having a user arranged in a first position;

FIG. 18B depicts the bulletproof vest of FIG. 18A having the user arranged in a second position;

FIG. 19 depicts a flow diagram for manipulating a flexible fabric;

FIG. 20A depicts an example flexible fabric having an array of size-changing materials;

FIG. 20B depicts an example size-changing material;

FIG. 20C depicts an example size-changing material;

FIG. 20D depicts an example size-changing material including shapeable materials;

FIG. 21A depicts a sealable sleep system with an adaptive actuation system;

FIG. 21B depicts a schematic view of the sealable sleep system of FIG. 21A; and

FIG. 22 depicts schematic view of a sealable sleep system with an adaptive actuation system and a temperature control system.

### DETAILED DESCRIPTION

The description that follows includes sample systems, methods, and apparatuses that embody various elements of the present disclosure. However, it should be understood that the described disclosure can be practiced in a variety of forms, in addition to those described herein.

The present disclosure describes systems and techniques for the adaptive actuation of a sleeping or resting structure, such as a flexible fabric or other material that is configured to engage a user, often for prolonged periods of time. A sample flexible fabric can be a component of a mattress or armchair, including a sleeping or resting structure configured to engage a user arranged in a sitting, prone, or semi-prone position. The mattress or armchair can be a smart mattress or furniture and can include an actuation assembly that is integrated with the flexible fabric in order to alter one or more characteristics of the sleeping or resting structure, including a firmness of the sleeping or resting structure. Sensors within or associated with the mattress, chair or sleeping system more generally can be configured to detect a condition of the user during use of the sleeping or resting structure. Position and pressure distribution of the user, as two examples, can be detected, and the actuation assembly can be configured to change characteristics of the sleeping or resting structure based on the detection. In some cases, the actuation assembly can be configured to alter the sleeping or resting structure in a manner that causes the user to gently move into a sitting, prone, or semi-prone position that encourages restful sleep. Audible and other conditions can also be detected during sleep. The actuation assembly can in turn be configured to gently move or roll a user to mitigate snoring, sleep apnea events during REM-sleep, and so on.

In one example, the actuation assembly can be configured to manipulate the sleeping or resting structure using a flexible bladder that contains a fluid and a pair of electrodes that operate to change the shape of the bladder. The flexible bladder and the pair of electrodes can form components of a Peano-HASEL actuator. The flexible bladder can be formed from an elastic material that defines a volume for a fluid therein. The pair of electrodes can be arranged with a first electrode positioned on a first side of the flexible bladder and a second electrode positioned on a second side of the flexible bladder. In operation, the pair of electrodes can move toward one another in response to an electrical charge. As the pair of electrodes move closer to one another, they can displace the fluid of the flexible bladder without operation of a separate pump, such as the power-intensive air pumps of conventional systems. The fluid displaced by the electrodes can cause the flexible bladder to deform, such as causing a portion of the flexible bladder to assume a larger dimension. As the flexible bladder deforms, the bladder can operate to press into the flexible fabric or other components of the actuation assembly in order to manipulate a sleeping or resting structure associated with the fabric.

Additionally or alternatively, the actuation assembly can implement various shapeable materials to manipulate the sleeping or resting structure. Broadly, as used herein, “shapeable material” can refer to any material that is configured for repeated deformation between a first configuration and a second configuration in response to energy from an energy source. The shapeable material can exhibit a memory effect so as to cycle between the first and second configurations based on one or more inputs from the energy source. Hundreds of thousands or even millions of cycles can be performed, often under heavy mechanical loads. In one example, the shapeable material can include a material responsive to a heat source. A shape memory alloy, including certain copper-aluminum-nickel alloys and nickel-titanium alloys can be used. Composites can also be used, including a blend of high-strength polymer fishing lines and sewing threads. Additionally or alternatively, the shapeable material can include a material that is responsive to a light source. Certain photopolymers or light-activated resins can be implemented that change properties when exposed to light, often in the ultraviolet or visible region.

The shapeable material can receive the heat energy and/or light energy and transition between the first and second configuration. The transitioning of the shapeable material between the first and second configurations can be adapted to manipulate the sleeping or resting structure and mattress more generally. In one example, the shapeable material can be arranged underneath a mattress and serve as a replacement for conventional wooden slats. The shapeable materials can be configured to become stiffer or softer with electrical stimuli, allowing for a responsive mattress without the need for large air cells or pumps. Additionally or alternatively, the shapeable materials can be integrated with the mattress itself in order to provide a more detailed (higher resolution) contouring zone. As one example, the shapeable material can define an arrangement of cilia-like structures that alternate shape based on the presence and receipt of light into the material. Individual ones of the cilia-like structure could in turn manipulate the sleeping or resting structure, allowing for fine-tuned control. Other arrangements of the shapeable material are contemplated and discussed herein.

In some examples, the actuation assembly can include various size-changing materials to manipulate the sleeping or resting structure. Broadly, as used herein, “size-changing materials” can refer to any materials that are configured for repeated transitions between a first size and a second size in response to energy from an energy source. In some examples, the size-changing materials can include materials responsive to a heat source, a light source, electrical stimuli, or the like. The size-changing materials can receive energy and transition between the first and the second sizes. In some examples, the size-changing materials may include any of the shapeable materials described herein, and the size-changing materials can change size due to differences in tension in the materials of the size-changing materials. In some examples, the size-changing materials can change size due to a difference in temperature. For example, the size-changing materials may have a volumetric coefficient of thermal expansion greater than about 50/° C. The transitioning of the size-changing materials between the first and the second sizes can be adapted to manipulate the sleeping or resting structure and mattress more generally. In some examples, the size-changing materials can be arranged underneath a mattress or cushion and serve as a replacement for conventional wooden slats. The size-changing materials can be configured to become stiffer or softer with electrical or other stimuli, allowing for a responsive mattress without the need for large air cells or pumps. Additionally or alternatively, the size-changing materials can be integrated with the mattress itself in order to provide a more detailed (higher resolution) contouring zone. As an example, the size-changing materials can define an arrangement of spherical structures that alternate size based on the presence and receipt of heat, light, other electrical stimuli, or the like into the materials. Individual ones of the spherical structures could in turn manipulate the sleeping or resting structure, allowing for fine-tuned control. Other arrangements of the size-changing materials are contemplated and discussed herein.

In some examples, the actuation assembly can include various phase change materials, such as phase change polymers (also referred to as phase transition polymers) to manipulate the sleeping or resting structure. Broadly, as used herein, “phase change materials” can refer to any materials that are configured for repeated transitions between different states of matter in response to energy from an energy source. In some examples, the phase change materials can include materials responsive to a heat source. The phase change materials can receive heat energy and transition between states, such as between a solid state and a liquid state. The transitioning of the phase change materials between states can be adapted to manipulate the sleeping or resting structure and armchairs and mattress more generally. In some examples, the phase change materials can be arranged underneath a cushion or mattress and serve as a replacement for conventional wooden slats. The phase change materials can be configured to become stiffer or softer with electrical or other stimuli, allowing for a responsive mattress without the need for large air cells or pumps. Additionally or alternatively, the phase change materials can be integrated with the mattress itself in order to provide a more detailed (higher resolution) contouring zone.

The actuation assembly can be implemented in a sleeping system. The sleeping system can include a mattress or armchair that uses the actuation assembly to modify one or more characteristics of the sleeping or resting structure, including firmness. More broadly, the sleeping system can be configured to provide total sensory immersion and adaptation to user-customizable settings, encouraging a restful sleep. Example systems include a pod structure that defines sleeping volume. The sleeping volume can include the mattress or armchair, support elements, and sufficient empty volume for the user to engage the mattress in a lying position and within the pod structure. The pod structure can include various sensors described herein to detect a condition of the user, including sensors that detect an audial input (responsive to snoring), a force or pressure input (responsive to user position and movement), a pulse input (responsive to heart rate), and others. As described herein, the actuation assembly can be responsive to the audial input, the force or pressure input, or the pulse input, such as altering a characteristics of the sleeping or resting structure, including altering the sleeping or resting structure in a way that gently rolls or manipulates the user. The sleeping structure can include further actuators or devices that alter the environment of the user in the sleeping volume, in response to the detected input. As illustrative examples, the sleeping structure can include vibratory devices to oscillate the mattress in a relaxing manner, audial device to introduce pleasing sounds to the sleeping volume, aroma generating devices to introduce pleasing smells into the sleeping volume, light devices to introduce tranquil and appropriately timed lighting, and so on.

In an example, the sleeping or resting structure can include actuators or devices to control and manipulate the physical vibration and movement of the structure to soundwaves, including from subsonic to hypersonic sound systems. In some examples the light devices can be controlled to manipulate light energy. The light energy can stimulate and support biorhythms (e.g., wavelengths of light can range in the red and infrared spectrums for transdermal cellular support or to shorter wavelengths in the blue/violet spectrum to stimulate dopamine production upon waking). In some examples, the K values of any white light can be manipulated to invoke sleep and waking states as required or preferred. For example, the light control of the sleeping or resting structure can include use a range of 1500-2000K for sleep support and 4000-6000K for waking. This can optimize circadian rhythm.

In some examples, the pod structure of the sleeping or resting system can be a pressure chamber or the like configured to provide an ambient pressure different from an atmospheric pressure. For example, the pod structure can be a pressure chamber configured to perform hyperbaric oxygen therapy or the like, may be configured to optimize an oxygen concentration provided to a user, or the like. In some examples, the pod structure can be configured to provide drugs or other medical therapies to a user during sleep, such as micro-dosing hallucinogenics to aid in focus, help with ADHD symptoms, or the like. The pod structure can include sensors to detect brainwaves, and can alter the sleeping or resting environment of the pod structure in response to detected brainwaves of a user. The sleeping system can be integrated with various user devices, such as a smartphone or other electronic device, to collect user data throughout the day. The sleeping system can then provide a tailored environment to a user based on daily activities, oxygen levels, heat rate, diet, consumables, environmental factors, and the like.

The actuation system of the present disclosure can also be implemented in a variety of flexible fabrics. As one example, the flexible fabric can be a component of a surgical table such as a surgical structure that supports a patient during a surgical operation. The actuation system can be configured to alter the flexible fabric in a manner that manipulates the patient during surgery. For example, the flexible fabric can be deformed by the actuation system in a manner that causes patient movement. The deformation can be tuned in a manner that moves the patient into a desired arrangement for surgery. The deformation can be part of preprogrammed sequence for a surgical operation and/or controlled by medical personnel during surgery. Easier access and surgical control can therefore be facilitated without necessarily relying on the direct physical contact with the patient by medical personnel during the surgery. Other applications of the flexible fabric and actuation system are contemplated and described herein, including application of the flexible fabric as a component of a medical table, a car seat, a garment, an insole, a bullet-proof vest, and so on.

Reference will now be made to the accompanying drawings, which assist in illustrating various features of the present disclosure. The following description is presented for purposes of illustration and description. Furthermore, the description is not intended to limit the inventive aspects to the forms disclosed herein. Consequently, variations and modifications commensurate with the following teachings, and skill and knowledge of the relevant art, are within the scope of the present inventive aspects.

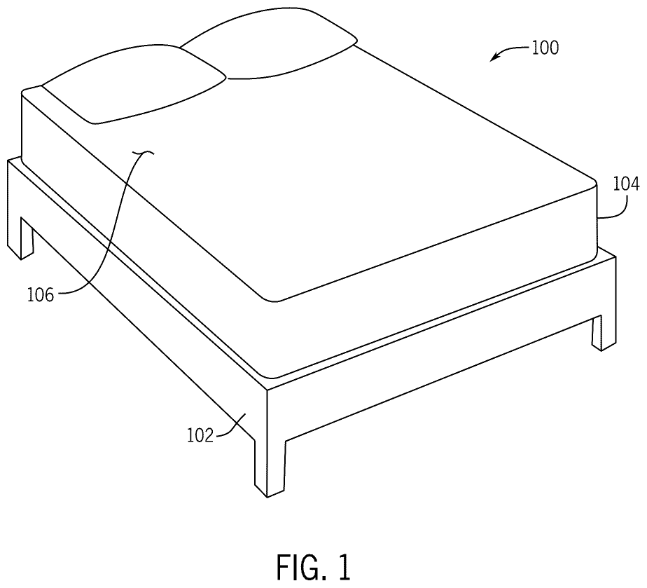

FIG. 1 depicts an example sleeping system 100. The sleeping system 100 is shown as including a frame support 102 and a mattress 104. The mattress 104 can define a sleeping or resting structure 106. The mattress 104 can be a smart mattress, such as those described generally above and as described in greater detail below. In this regard, the mattress 104 can include an actuation assembly that is adapted to alter one or more characteristics of the sleeping or resting structure 106. For example, the actuation assembly can alter a firmness of the sleeping or resting structure 106. The sleeping or resting structure 106 can be formed from or as a flexible fabric or other material is configured to engage a user in a sitting, prone, or semi-prone position. The sleeping or resting structure 106 can therefore be adapted to deform in response to the configuration of the actuation assembly. In some cases, the actuation assembly can operate to cause to the sleeping or resting structure 106 to deform in a manner that causes the user to gently roll or otherwise gradually reposition the user on the sleeping or resting structure 106 for a restful sleep.

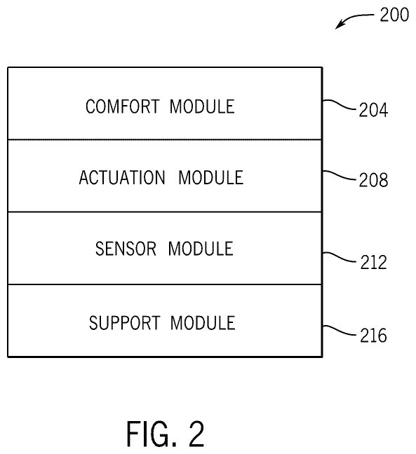

FIG. 2 depicts a functional diagram of an adaptive actuation assembly 200. The adaptive actuation assembly 200 can be implemented in the sleeping system 100 of FIG. 1. The adaptive actuation assembly 200 is shown functionally in FIG. 2 as including a comfort module 204, an actuation module 208, a sensor module 212, and a support module 216. The comfort module 204 can include any appropriate material to facilitate engagement of the adaptive actuation assembly 200 with a user. In the case of the mattress 104, the comfort module 204 can include the sleeping or resting structure 106 and components of the mattress 104 that collectively define the sleeping or resting structure 106 for engaging the user in a sitting, prone, or semi-prone position. For example, the comfort module 204 can include a cushion, foam, fabric, sheet, or other material that is configured to enhance the comfort of the user on the sleeping or resting structure 106. In one implementation, an ultra-suede topped memory foam cushion can be used. The comfort module 204 can additionally or alternatively include a flexible fabric, such as any of the flexible fabrics described herein. For example, the flexible fabric can be sufficiently flexible to allow for repeated deformation by actuators of the actuation assembly 200. The flexible fabric can also be sufficiently robust to withstand the weight of the user and/or the force from the actuators without undue wear or breaking. The flexible fabric and the comfort module 204 more generally can define an interface for the actuators of the actuation assembly 200. In some cases, the actuators can be held at least partially within and/or enmeshed within the flexible fabric itself, allowing the various actuators to be positioned in relatively close proximity to the sleeping or resting structure 106.

With respect to the actuation module 208, the actuation assembly 200 can include various actuator devices, assemblies, sub-assemblies, and so on to facilitate the manipulation of the comfort module 204. As described herein, the actuation module 208 can include a Peano-HASEL-type actuator device. For example, the actuation module 208 can include a flexible bladder that contains a fluid. A pair of electrodes can be separated from one another by the flexible bladder. The pair of electrodes can be operable to receive an electrical charge that causes movement of the electrodes toward one another. The movement of the electrodes toward one another can displace the fluid and deform the bladder. The actuator can be engaged with the comfort module 204 in a manner such that deformation of the bladder causes the manipulation of the comfort module 204. As one example, a portion of the flexible bladder can be enlarged and pressed against a portion of the flexible fabric.

The actuation module 208 can also include various shapeable materials. The shapeable materials, as described above, can include materials that are adapted to change shape in response to the receipt of energy, such as the receipt of energy from heat and/or light source. The shapeable materials can exhibit a memory effect, allowing the shapeable materials to deform and return to an un-deformed shape in a consistent manner. Shape memory alloys, nylons, photopolymers can be included in the shapeable materials. The shapeable materials in some cases can be integrated with the comfort module 204 such that the deformation of the shapeable material causes an associated deformation of the flexible fabric or other material. Additionally or alternatively, the shapeable materials can be integrated with the support module 216, described below, to provide structural support and firmness control to the system.

In some examples, the actuation module 208 can include various size-changing materials. The size-changing materials, as described above, can include materials that are adapted to change size in response to the receipt of energy, such as the receipt of energy from a heat source, a light source, other electrical stimuli, or the like. The size-changing materials can change sizes in a consistent manner in response to the receipt of energy. The size-changing materials can include materials having volumetric coefficients of thermal expansion greater than about 20/°, greater than about 30/°, greater than about 50/°, greater than about 100/°, or the like. In some examples, the size-changing materials may include the shapeable materials, arranged to form desired shapes. For example, the shapeable materials may be arranged to form spheres, polyhedrons, cylinders, cones, or any other suitable shapes, thus forming the size-changing materials. The size-changing materials can be integrated with the comfort module 204 such that the deformation of the size-changing materials causes an associated deformation of the flexible fabric or other material. Additionally or alternatively, the size-changing materials can be integrated with the support module 216, described below, to provide structural support and firmness control to the system.

In some examples, the actuation model can include phase change materials, such as phase change/transition polymers. The phase change materials, as described above, can include materials that are adapted to change to a different state of matter in response to the receipt of energy, such as the receipt of energy from a heat source. In some examples, the phase change materials can transition between solid and liquid states in a consistent manner in response to the receipt of energy. The phase change materials can be integrated with the comfort module 204 such that the transitioning of the phase change materials causes an associated deformation of the flexible fabric or other material. Additionally or alternatively, the phase change materials can be integrated with the support module 216, described below, to provide structural support and firmness control to the system.

With respect to the sensor module 212, the actuation assembly 200 can implement a variety of sensors that detect a condition of the user. The condition of the user can be used to control one or more of the actuators of the actuation module 208. In one example, the electrodes of the actuator (e.g., Peano-HASEL actuators) can be used to detect a movement and/or pressure distribution of the user. For example, an initial capacitance can be defined between the electrodes. The position of the electrodes relative to one another can shift in response to movement of the user, thereby altering the capacitance between the electrodes. The change in capacitance can be detected and correlated to a force input that cause the electrodes to move. Where the force input exceed as threshold, the actuation assembly 200 can operate one or more of the actuators in order to change a characteristic of the mattress, such as adjusting the firmness. In some cases, the force input can be determined at each of an array of electrodes and analyzed to define a pressure distribution of the user on the mattress 104. The pressure distribution can be analyzed in order to determine the position of the user on the sleeping or resting structure 106, and tracked over time to determine sleeping patterns of the user.

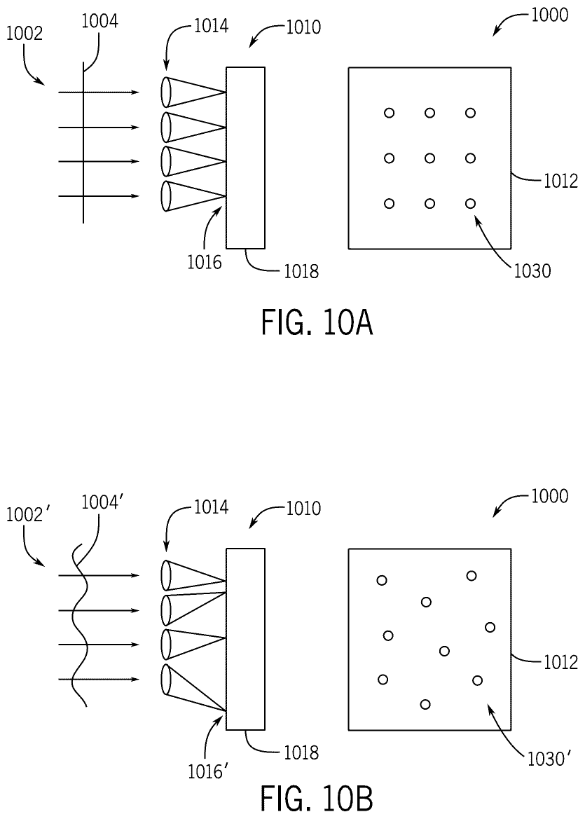

Additionally or alternatively to the electrodes of the actuators, the sensor module 212 can also include other sensors to detect a force input or other inputs. Resistance-based switches can be used, for example. A wavefront or optical sensor can also be used to detect pressure distribution of the user, as described in greater detail with respect to FIGS. 10A and 10B. Additional sensors can be implemented in order to detect audial inputs, such as snoring. In this regard, the actuation assembly 200 can manipulate the sleeping or resting structure 106 in response to a user snoring. This can include gently rolling the user and potentially mitigating the effects of sleep apnea during REM-sleep. Light and vibration sensors can also be implement and integrated with the various actuators described herein in order to manipulate the sleeping or resting structure 106.

In some examples, the sensor module 212 can be integrated with external electronic devices and/or sensors, which may collect user data throughout the day. For example, the sensor module 212 may be integrated with a user's smartphone or other electronic device, and may collect data relevant to a user's experience and condition throughout the day. The data may include daily activities, oxygen levels, heat rate, diet, consumables, environmental factors, location, and any other data relevant to a condition of the user. The actuation assembly can manipulate the sleeping or resting structure 106 in response to the user data to provide an optimal sleep experience. Moreover, as will be discussed in detail, the user data of the sensor module 212 can be provided to additional systems of a sleeping system. For example, the user data may be provided by the sensor module 212 to an ambient effect module to provide optimized ambient air to the user, such as by altering a gaseous concentration and pressure of ambient air provided to the user.

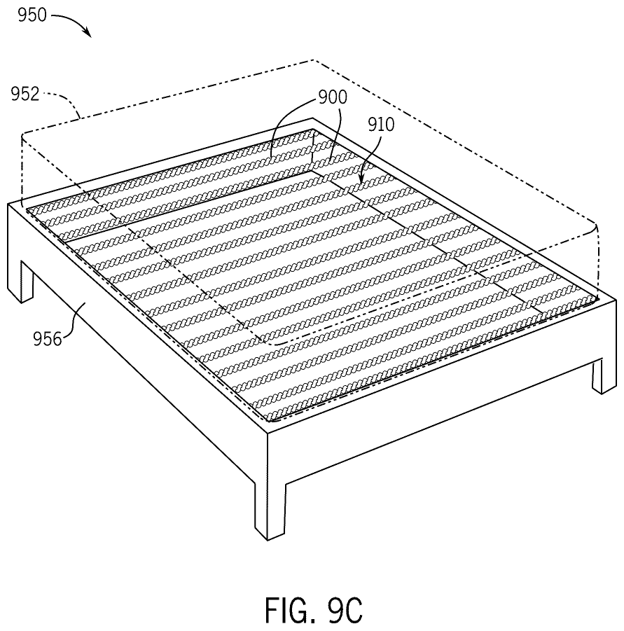

With respect to the support module 216, the actuation assembly 200 can include structural features that operate to support the user in the sitting, prone, or semi-prone position on the sleeping or resting structure 106. In some cases, the support module 216 can include structural elements, such as a support frame. Additionally or alternatively, the support module 216 can include a waterbed chamber, foams, cushions, and so on that provide additional support and comfort to the user. The various actuators of the present disclosure can, in some cases, form components of the support module 216. As one example, the shapeable materials, the size-changing materials, and/or the phase change materials, described herein can used to define a series of support slats underneath the mattress. The shapeable materials, the size-changing materials, and/or the phase change materials can thus support the weight of a user engaged on the sleeping or resting structure 106 and be arranged to alter characteristics of the mattress 104, such as deforming in a manner that imparts additional stiffness to the mattress 104 as needed and as shown in greater detail in FIG. 9C.

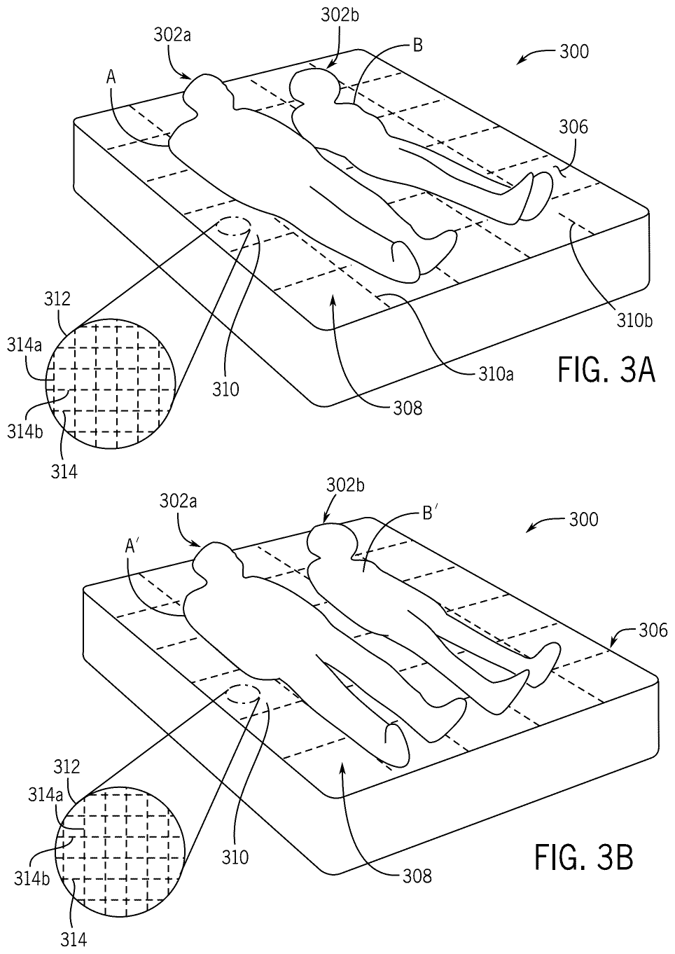

With reference to FIGS. 3A and 3B, a mattress 300 is illustrated that can implement the actuation assembly 200 described above. The mattress 300 can include a sleeping or resting structure 306, including or being defined in part by a flexible fabric. The sleeping or resting structure 306 is shown as having an actuation grid 308 (phantom lines). The actuation grid 308 can divide the sleeping or resting structure 306 into representative portions 310. Broadly, the actuation assemblies described herein can be adapted to alter characteristics of the mattress 300 in a manner that is customized for each representative portion 310. For example, the actuation assembly 200 can be integrated below the sleeping or resting structure 306 and can be configured to manipulate each representative portion 310. In some cases, the actuation assembly 200 can manipulate each representative portion 310 individually. For example, a first representative portion 310a can be manipulated in a first manner by the actuation assembly 200. A second representative portion 310b can be manipulated in a second, optionally, different manner by the actuation assembly 200. For the sake of illustration, the first manner of manipulation can cause the sleeping or resting structure 306 to have a first stiffness at the first representative portion 310a and the second manner of manipulation can cause the sleeping or resting structure 306 to have a second stiffness at the second representative portion 310b. This can help establish and maintain a comfort level of different users on the same sleeping or resting structure 306, such as a first user 302a in a position A and a second user 302b in a position B, as shown in FIG. 3A.

It will be appreciated, however, that the representative portions 310 are shown for purposes of illustration. The actuation assemblies of the present disclosure can allow for ultra-fine-tuned control of characteristics of the sleeping or resting structure 306. In this regard, FIG. 3A further shows a precision grid 312 (phantom lines) having representative portions 314. The representative portions 314 can represent subsets of the macro representative portions 310. Each representative portion 314 can be configured for manipulation by the actuation assembly 200. In some cases, this can include manipulating a first representative portion 314a in a first manner and a second representative portion 314b in a second, optionally different manner. The micro control at the precision grid 312 can allow for customized manipulations of the sleeping or resting structure 306 to influence movement, position, and condition of the user. As one example, the representative portions 314 can have varying stiffness and/or deformations from a baseline in order to maintain the user in a supportive arrangement on the sleeping or resting structure 306. Additionally or alternatively, the manipulations of the representative portions 314 can be used to move or reposition the user, such as by gently repositioning the user into a position that supports a restful sleep. For example, as shown in FIG. 3B, the first user 302a is arranged in a position A′ and the second user 302b is arranged in a position B′ as a result of the manipulation of the sleeping or resting structure 306 by the actuation assembly 200.

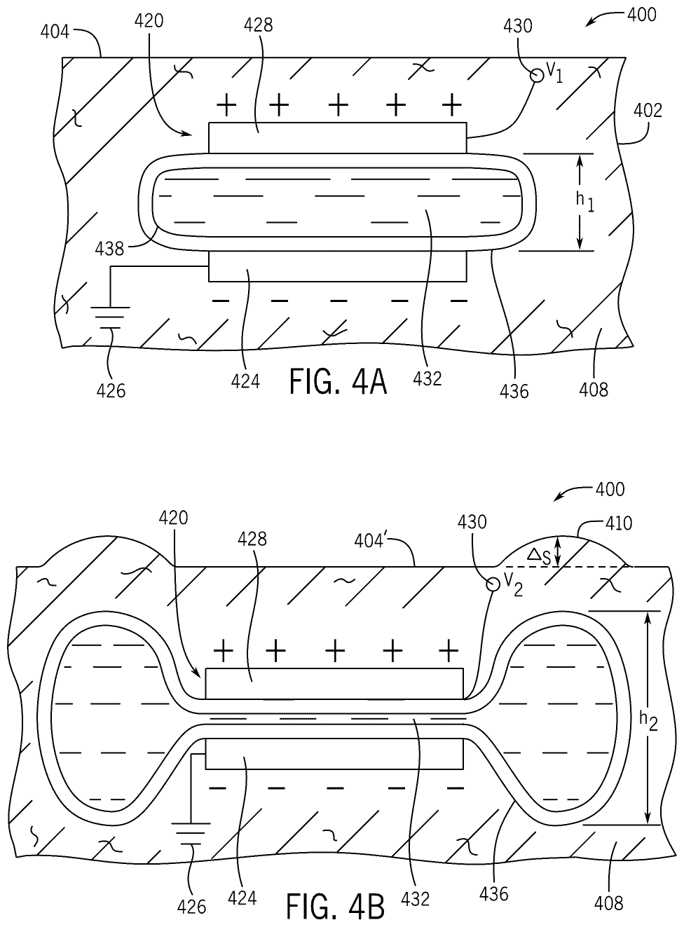

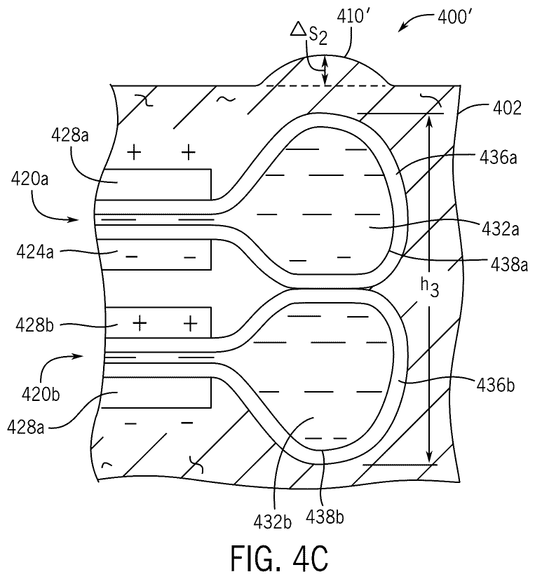

The actuation assemblies described herein can be implemented with a flexible fabric. With respect to FIGS. 4A-4C, a flexible fabric 400 is shown having an actuation assembly 420. The flexible fabric 400 can be substantially analogous to the flexible fabrics described above with respect to the comfort module 204. The flexible fabric 400 can be a component or define a sleeping or resting structure of a mattress. In the example of FIGS. 4A-4C, the flexible fabric 400 includes a flexible outer layer 402, a flexible surface 404, and a fabric region 408. The flexible outer layer 402 can generally be adapted to define an interface with the actuation assembly 420. For example, as shown schematically in FIG. 4A, the actuation assembly 420 can be held within or more generally interface with the fabric region 408. The actuation assembly 420 can operate to manipulate the fabric region 408 and cause deformations of the flexible surface 404.

In the example of FIGS. 4A-4C, the actuation assembly 420 can include a Peano-HASEL actuator or other actuation device that displaces fluid of a flexible bladder using electrodes. The actuation assembly 420 is shown in FIGS. 4A-4C as including a first electrode 424 and a second electrode 428. The first and second electrodes 424, 428 can collectively define a pair of electrodes. The second electrode 428 can be connected to a current supply 430. The first electrode 424 can be connected to a ground 426. A flexible bladder 436 defining a cavity 438 and containing a fluid 432 can be positioned between the first and second electrodes 424, 428.

In operation, FIG. 4A depicts the actuation assembly 420 in a first or unactuated configuration. In the unactuated configuration, the first and second electrodes 424, 428 can be generally spaced apart from one another. The flexible bladder 436 can be in a relaxed or substantially un-deformed shape. In the first configuration, the flexible bladder can have a height h₁. When the flexible bladder has the height h₁, the flexible outer layer 402 can be substantially un-deformed, as shown in FIG. 4A.

The actuation assembly 420 can be configured to manipulate the flexible outer layer 402. In operation, the actuation assembly 420 can receive an electrical charge at the first and second electrodes 424, 428. The electrical charge can bias the first and second electrodes 424, 428 to move closer to one another, as shown in FIG. 4B. The first and second electrodes 424, 428 can move toward one another and press towards the flexible bladder 436 that is positioned therebetween. The fluid 432 contained within the flexible bladder 436 is displaced with the flexible bladder 436 by the movement of the first and second electrodes 424, 428, such as being displaced towards ends of the flexible bladder 436 in which no electrodes are present. The displacement of the fluid 432 can cause the flexible bladder 436 to stretch or deform. For example, the fluid 432 can be displaced toward an end portion of the flexible bladder 436 in which no electrodes are present. This can cause the flexible bladder 436 to have a deformed or modified shape. In the example of FIG. 4B, the flexible bladder 436 is deformed such that an end of the flexible bladder 436 has a height h₂. The height h₂ can be greater than the height h₁. The value of the second height h₂ can be based on a value of the electrical charge applied to the first and second electrodes 424, 428. In this regard, the value of the second height h₂ can be controlled using the applied electrical charge, thereby allowing for controlled deformation of the actuation assembly 420 to a predetermined and/or customizable value.

The flexible surface 404 can be deformed by transitioning the flexible bladder 436 from the first height h₁ to the second height h₂. For example, transitioning of the flexible bladder 436 to the second height h₂, can cause a portion of the flexible surface 404 to be deformed, such as being deformed in a manner that defines a modified contour 404′ shown in FIG. 4B. The modification can be locally confined to a manipulated portion 410 of the flexible surface 404. The modified contour 404′ can have a deformed height Δₛ. The deformed height \, can correspond to a value of the change in height between the second height h₂ and the first height h₁. In this regard, the value of the deformed height Δₛ can be controlled using the applied electric charge, which, as described above, is used to control the value of the second height h₂. Accordingly, the magnitude of the deformation of the flexible surface 404 can be fine-tuned to provide a surface manipulation that is configured to address a specified user condition, such as user repositioning, spinal support, sleep apnea mitigation, and so on.

It will be appreciated that the flexible fabric 400 of FIGS. 4A and 4B is shown with a single actuation assembly 420 for purposes of illustration. Multiple actuation assemblies can be implemented with the flexible fabric 400. The multiple actuation assembles can cooperate with one another to produce combinative or additive effects for the manipulation of the flexible fabric 400. For purposes of illustration, FIG. 4C shows a flexible fabric 400′. The flexible fabric 400′ has a first actuation assembly 420a and a second actuation assembly 420b. The first and second actuation assemblies 420a. 420b can be substantially analogous to the actuation assembly 420 described above in relation to FIGS. 4A and 4B. In this regard, the first actuation assembly 420a can include a first electrode 424a, a second electrode 428a, a fluid 432a, a flexible bladder 436a, and a cavity 438a. Further, the second actuation assembly 420b can include a first electrode 424b, a second electrode 428b, a fluid 432b, a flexible bladder 436b, and a cavity 438b. The first and second actuation assemblies 420a, 420b can be stacked on top of one another and integrated with the fabric region 408 of the flexible outer layer 402.

FIG. 4C shows the first and second actuation assemblies 420a, 420b in a configuration in which an electrical charge is applied to the respective electrodes. In this regard, the first and second electrodes 424a, 428a can move toward one another and displace the fluid 432a and deform a portion of the flexible bladder 436a. Further, the first and second electrodes 424b, 428b can move toward one another and displace the fluid 432b and deform a portion of the flexible bladder 436b. The deformed portions of the flexible bladders 436a, 436b can cooperate with one another to define a deformed height h₃ of the collective actuation assemblies. The flexible outer layer 402 can be deformed when the first and second actuation assemblies 420a, 420b exhibit the height h₃. For example, the first and second actuation assemblies 420a, 420b having the height h₃ can be configured to cause the flexible outer surface 404 to have a manipulated portion 410′ that exhibits a deformed height Δs₂. A value of the deformed height Δs₂ can correspond to a value of the deformed height h₃ of the flexible bladders 436a, 436b. In some cases, the presence of multiple flexible bladders can produce an additive or a multiplicative effect on the value of the deformed height Δs₂. For example, the flexible bladder 436a can encounter resistance from the flexible bladder 436b upon the displacement of fluid by the electrodes. As such, the flexible bladder 436a can be biased toward deforming to a greater extent in a direction extending toward the flexible outer surface 404, which can present less resistance to the expansion of the flexible bladder 436a, thereby allowing for the enhanced deformation at the flexible outer surface 404 than may otherwise be possible with a single bladder.

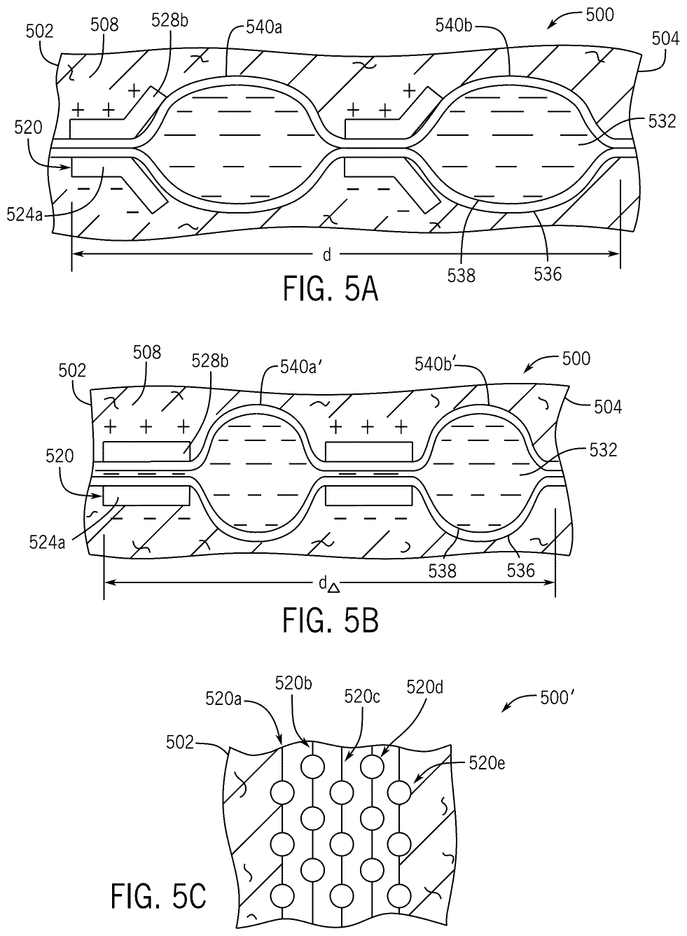

With respect to FIGS. 5A and 5B, a flexible fabric 500 is shown with an actuation assembly 520. The flexible fabric 500 can include a flexible outer layer 502, a first end 504, and a fabric region 508. The actuation assembly 520 can be substantially analogous to the actuation assembly 420 and can include a flexible bladder 536 that contains a fluid 532 in a cavity 538. The fluid 532 can be displaced by the operation of the actuation assembly 520 in order to manipulate the flexible outer layer 502.

Notwithstanding the foregoing similarities, the actuation assembly 520 includes a first pair of electrodes and a second pair of electrodes. For example, the actuation assembly 520 includes electrodes 524a, 528a that define the first pair of electrodes and electrodes 524b, 528b that define the second pair of electrodes. The flexible bladder 536 can define the cavity 538 as a continuous cavity that extends between the electrodes 524a, 528a of the first pair of electrodes and the electrodes 524b, 528b of the second pair of electrodes. As shown in FIG. 5A, interposed with the first and second pairs of electrodes, the flexible bladder 536 can define a first bulbous portion 540a and a second bulbous portion 540b. The continuous cavity 538 can have a length d in the unactuated configuration shown in FIG. 5A.

In operation, the actuation assembly 520 can be adapted to receive an electrical charge that causes the electrodes 524a, 528a of the first pair of electrodes to move closer to one another. The actuation assembly 520 can be further adapted to receive an electrical charge that causes the electrodes 524b, 525b of the second pair of electrodes to move closer to one another. As shown in FIG. 5B, the movement of the respective electrodes of the first and second pairs of electrodes can cause the fluid 532 to be displaced in order to deform the flexible fabric 500. In some cases, as shown in FIG. 5C, the deformation of the flexible bladder 536 can cause the length of the continuous cavity 538 to contract, such as contracting to a length da. The actuation assembly 520 can be integrated with the fabric region 508 in a manner such that the flexible outer layer 502 is manipulated or deformed when the flexible bladder 536 has the length da. For example, the contraction of the flexible bladder 536 to the length da can cause the first end 504 of the flexible fabric 500 to be pulled or retracted by a corresponding amount. The amount of retraction of the first end 504 can be tuned according to the electrical charge applied to the electrodes, as described above with respect to FIGS. 4A-4C.

In some cases, the flexible fabric 500 can include multiple actuation assemblies arranged in parallel. For example, FIG. 5C depicts a flexible fabric 500′ having a first actuation assembly 520a, a second actuation assembly 520b, a third actuation assembly 520c, a fourth actuation assembly 520d, and a fifth actuation assembly 520e. Each of the actuation assemblies 520a-520e can be analogous to the actuation assembly 520 described above with respect to FIGS. 5A and 5B. In this regard, each of the actuation assemblies 520a-520e can have a flexible bladder that defines a continuous cavity that extends between multiple electrodes. The flexible bladders of each of the actuation assemblies 520a-520e can therefore be configured to contract along a lengthwise dimension in response to an electrical charge. The arrangement of multiple actuation assemblies in series can contribute to additive or multiplicative effects with respect to a value of deformation of the flexible outer layer 502. For example, the presence of multiple actuation assemblies can cause a localized portion of the flexible outer layer to be deformed with a great amount of force, the application of which is more finely controlled, including individually at each of the actuation assemblies 520a-520e. This can be beneficial for a range of applications, such as using the flexible fabric 500′ to reposition a sleeping user in a gentle manner.

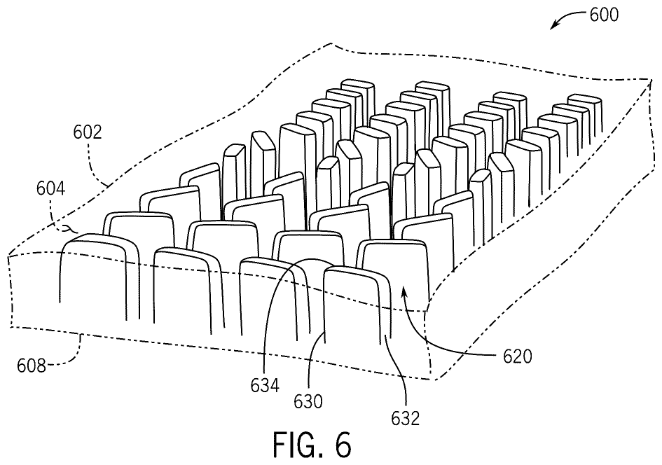

The actuation assemblies of the present disclosure can also include shapeable materials, as described herein. For example and with reference to FIGS. 6-9C, various actuation assemblies are shown in which a shapeable material is used to manipulate a portion of a flexible fabric. The shapeable material can be configured for repeated cycling between a first shape and a second shape. The shapeable material can be configured to transition between the first shape and the second shape upon the receipt of energy from an energy source, including receiving heat and/or light energy. The shapeable material can have memory effects so as to return to first shape upon the cessation of the energy.

Turning to FIG. 6, an example flexible fabric 600 is shown having an actuation assembly 620. The actuation assembly 620 includes a collection of shapeable materials and components configured to manipulate the flexible fabric 600. The flexible fabric 600 is shown in FIG. 6 as including a fabric outer layer 602, a flexible surface 604, and a fabric region 608. The actuation assembly 620 can be held at least partially within or otherwise interface with the fabric region 608. The actuation assembly 620 is operable to cause a deformation of the flexible outer surface 604 upon actuation.

To facilitate the foregoing, the actuation assembly 620 can include an array of shapeable components, including a shapeable component 630. The shapeable component 630 can have a first end 632 that can be fixed or otherwise attached to a base. The shapeable component 630 can have a second end 634 that is a free end opposite the first end 632. The shapeable component 630 can be formed at least partially from a shapeable material, such as any of the shapeable materials described herein. In this regard, the shapeable component 630 can be configured to transition between a first configuration and a second configuration upon the receipt of energy. In the example of FIG. 6, the transition of the shapeable component 630 can cause the second end 634 to move relative to the fabric region 608. The movement of the second end 634 relative to the fabric region 608 can be configured to cause a deformation of the flexible surface 604.

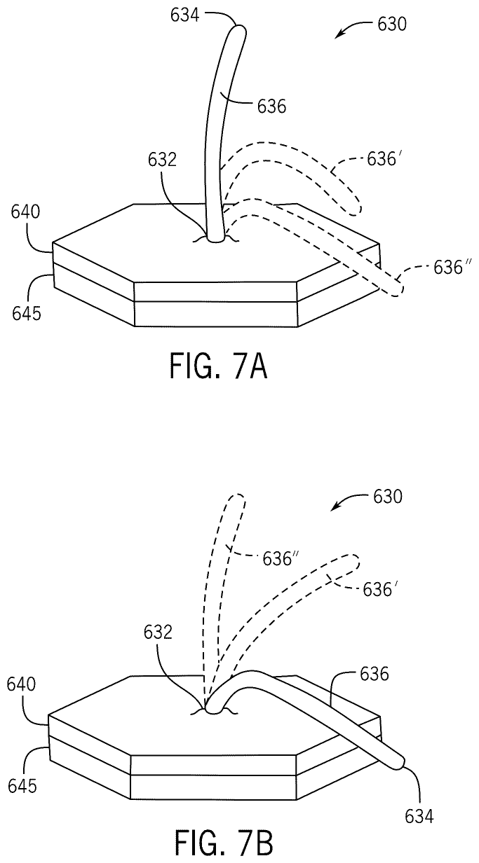

With reference to FIGS. 7A and 7B, the shapeable component 630 is shown associated with a base 640 and an energy source 645. The shapeable component 630 can be connected to the base 640 at the first end 632. The energy source 645 can be coupled with the base 640 and/or the shapeable component 630 and be operable to deliver energy to the shapeable component 630. For example, the shapeable component 630 can be or include a photopolymer or light-activated resin that changes properties when exposed to light. The energy source 645 can include a light source that is configured to deliver light to the shapeable component 630 and cause the shapeable component 630 to change shape.

In the configuration of FIG. 7A, the shapeable component 630 is shown as having a first body arrangement 636. For example, in the first body arrangement 636, the shapeable component 630 can substantially extend and protrude from the base 640. The first body arrangement 636 can correspond to a state of the actuation assembly 620 in which the shapeable component 630 receives light energy from the energy source 645. FIG. 7A also shows a second body arrangement 636′ and a third body arrangement 636″. The second body arrangement 636′ and the third body arrangement 636″ can correspond to a configuration of the shapeable component 630 in which the shapeable component receives less or no light from the energy source 645. In this regard, the shapeable component 630 can be configured to transition between a range of configurations and positions between the first body arrangement 636 and the third body arrangement 636″ based on a quantity of light received from the energy source 645. For example and as shown in FIG. 7B, the shapeable component 630 can exhibit the third body arrangement 636″ when the energy source 645 emits substantially no light toward the shapeable component 630.

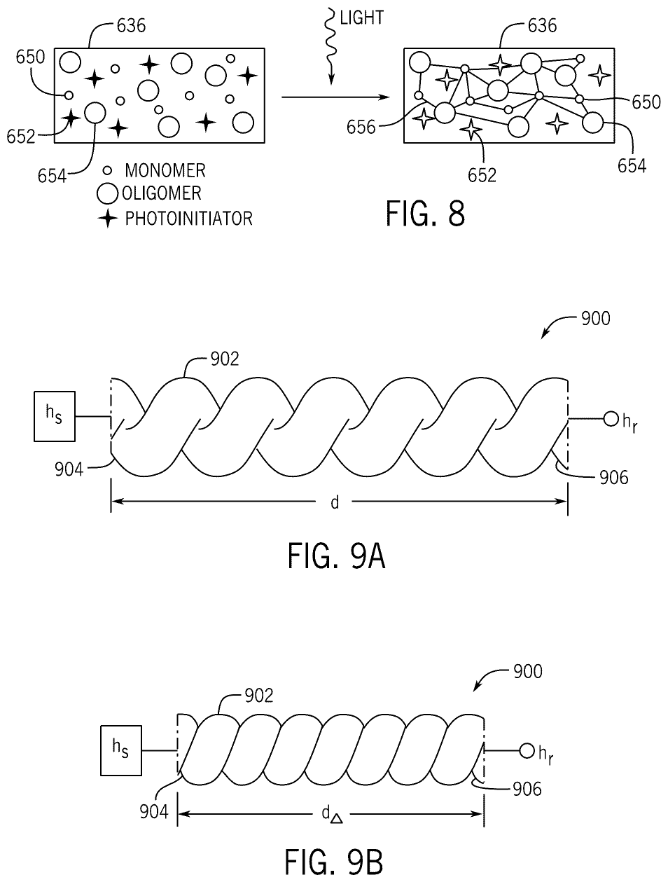

In one example, the shapeable component 630 can be formed at least partially from a photopolymer or light activated resin. In this regard, FIG. 8 shows a schematic diagram of the photopolymer or light activated resin that permits the shapeable component 630 to transition shapes. As shown in FIG. 8, in the third body arrangement 636″ the shapeable component 630 generally includes a monomer 650, an oligomer 652, and a photoinitiator 654. The monomer 650, the oligomer 652, and the photoinitiator 654 can be associated with one another to define the third body arrangement 636″, as shown in FIG. 7B. The shapeable component 630 can be exposed to light in order to transition the shapeable component 630 to the first body arrangement 636. As shown in FIG. 8, upon the receipt of light, links 656 can be formed between at least some of the monomer 650, the oligomer 652, and the photoinitiator 654. In some cases, the formation of the links 656 can cause one or more material properties of the shapeable component 630 to be altered, including causing a portion of the shapeable component 630 to contract or otherwise change in length such that the shapeable component 630 is operable to transition between a first configuration and a second configuration. In some cases, upon the cessation of the light, the links 656 can dissipate and allow the shapeable component 630 to transition from the first body arrangement 636 to the third body arrangement 636″. Further, the links 656 can be configured to dissipate in a manner that allows the shapeable component 630 to exhibit the memory effect and produce the shapeable component 630 in substantially the same shape initial shape.

In other examples, the various actuation assemblies of the present disclosure can include a shapeable material that is manipulatable upon the receipt of heat energy. For example, Joule heating can be used to heat a material, including nylon or certain alloys, to change a shape of the shapeable material. With reference to FIGS. 9A and 9B, a sample shapeable material 900 is shown that can be configured to alternate between a first shape and a second shape upon the receipt of heat. The shapeable material 900 can define a coiled structure 902 having a first end 904 and a second end 906. The first end 904 can be associated with a heat source hₛ. The second end 906 can be associated with a heat output hr. The heat source hₛ can be an electrical current source. The heat output hr can be an output of the electrical current subsequent to the current passing through the coiled structure 902. The coiled structure 902 can have a length d in a first configuration, such as that shown in FIG. 9A. The first configuration can correspond to a substantially cool configuration in which a reduced amount of heat energy, including no heat energy, is introduced to the shapeable material 900.

In a second configuration, heat energy can be introduced to the shapeable material 900 in order to manipulate the coiled structure 902. For example and as shown in FIG. 9B, heat energy can be introduced to the coiled structure 902 via the heat source hₛ. The introduction of heat to the coiled structure 902 can cause the coiled structure 902 to exhibit a reduced length d_{Δ}. The reduced length da can be at least 90% of the length d, at least 70% of the length d, at least 50% of the length d. The value of the reduced length d can be partially based on the amount of heat energy added to the coiled structure 902. For example, in a first instance, a first quantity of heat can be introduced to the coiled structure 902 by the heat source hₛ in order to reduce the length of the coiled structure 902 by a first amount. In a second instance, a second, greater quantity of heat can be introduced to the coiled structure 902 by the heat source hₛ in order to reduce the length of the coiled structure 902 by a second, greater amount.

The shapeable material 900 can be used to manipulate a flexible fabric, such as that of a mattress. In one example, shown in FIG. 9C, the shapeable material 900 can be incorporated into a sleeping system 950 in order to support a mattress 952 (shown in phantom). For example, the shapeable material 900 can form a series of slats 910 in a base frame 956. The series of slats 910 can be configured to function as several responsive hammocks underneath the mattress 952. For example, each shapeable material 900 can be adapted to receive electrical stimuli to change an effective length of the coiled structure 902, as described above. The change in the effective length can make sections of the slats 910 stiffer or softer. The changing stiffness of the slats 910 could in turn alter a stiffness of the mattress 952, or otherwise contour the mattress 952 to the individual needs of the user.

In some examples, a wavefront sensor can be used to detect a position, orientation, and/or movement of a user relative to a flexible fabric. For example, a wavefront sensor can be configured to detect a perturbed wavefront. The perturbed wavefront can be associated with a movement of a user. One or more processing units can measure a value of the perturbed wavefront and associate the perturbed wavefront with the user's movements.

FIGS. 10A and 10B depict an example sensing system 1000 that measures a perturbed wavefront. The sensing system 1000 can include a wave-front sensor 1010, a lens array 1014, a sensing structure 1018, and a focal point landing surface 1012. The system 1000 can operate by receiving incoming light 1002 at the lens array 1014. The incoming light 1002 can collectively define a wave front 1004. The lens array 1014 can concentrate and direct the light toward focal points 1016 on the sensing structure 1018. The focal points 1016 can establish a focal point arrangement 1030 along the focal point landing surface 1012. In the example of FIG. 10A, the wave front 1004 can be substantially unperturbed. In this regard, the focal point arrangement 1030 is shown in FIG. 10A as forming a grid configuration of substantially even spacing.

With respect to FIG. 10B, the wave-front sensor 1010 can receive incoming light 1002′. The incoming light 1002′ can define a perturbed wavefront 1004′. For example, the incoming light 1002′ can be received by the lens array 1014 at different incident angles than the incoming light 1002 of FIG. 10A. In this regard, the lens array 1014 can operate to direct the incoming light toward focal points 1016′ on the sensing structure 1018. The focal points 1016′ can establish a focal point arrangement 1030′ along the focal point landing surface 1012. In the example of FIG. 10B, the wave front 1004′ can be substantially perturbed. Accordingly, the focal point arrangement 1030′ is shown in FIG. 10B as forming a configuration of substantially uneven spacing. The wavefront sensor 1010 can be operative to measure a deviation of the focal point arrangement 1030′ from the focal point arrangement 1030 in order to determine a magnitude of the perturbed wavefront relative to a baseline. The magnitude of the perturbed wavefront can in turn be correlated to a movement of the user relative to the flexible fabric.

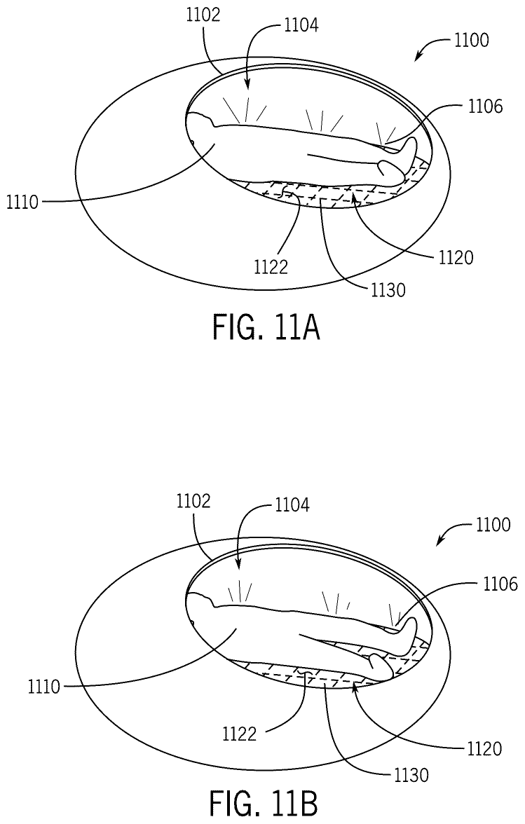

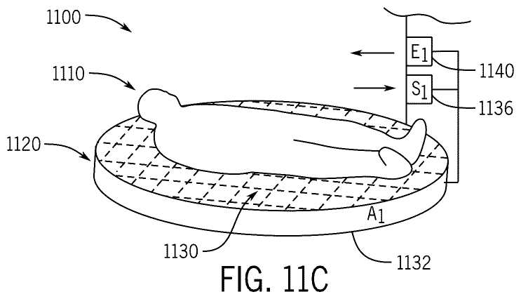

FIGS. 11A-18B depict example implementations of the flexible fabrics and actuation assemblies described herein. Broadly, the actuation assemblies of the present disclosure can be used to manipulate substantially any flexible fabric that is configured to engage a user. For example, a flexible fabric can be configured to engage a user for a prolonged period of time (e.g., sleeping), to support a user in a predetermined position (e.g., sitting or standing), to remain with a user during movement (e.g., clothing), and so on. The actuation assemblies described herein can be configured to manipulate the flexible fabric in a manner that causes the flexible fabric to deliver a force input to the user. For example, the manipulation of the flexible fabric can deform an outer surface of the flexible fabric and the user can receive a force input corresponding to the deformation of the flexible fabric. In some cases, the deformation can be sufficient to reposition the user in order to adapt the user to dynamic and measurable conditions.

With reference to the embodiment of FIGS. 11A-11C, a sleeping system 1100 is shown. The sleeping system 1100 can implement an adaptive actuation system, such as that described above, to control one or more characteristics of a mattress based on a user condition. The sleeping system 1100 can be adapted to contribute to an immersive sleeping experience. For example, the sleeping system 1000 can include a pod 1102 that defines a sleeping volume 1104. The sleeping volume 1104 can have ambient effects 1106, such as lights, smells, sounds, and the like. The sleeping volume 1104 can be configured to hold a mattress 1120 and a user 1110 therein. The pod 1102 can be substantially spherical in shape and form a partial enclosure over the user 1110. The mattress 1120 can include a sleeping or resting structure 1122 that is configured to engage the user in the sitting, prone, or semi-prone position within the sleeping volume 1104. The mattress 1120 can include an adaptive actuation system 1130, such as any of the adaptive actuation systems described herein. The adaptive actuation system 1130 is shown in the example of FIG. 11A with a representative grid (shown in phantom). The adaptive actuation system 1130 can be configured to modify the sleeping or resting structure 1122, as described herein. For example, in the first configuration shown in FIG. 11A, the adaptive actuation system 1130 can operate to maintain the user 1110 in a first position. The adaptive actuation system can further operate to manipulate the sleeping or resting structure 1122 and move the user 1110 to a second position, as shown in FIG. 11B.

The sleeping system 1100 can be configured to detect a condition of the user 1110 and alter one or more characteristics of a sleeping experience. In the schematic diagram of FIG. 11C, the user 1110 is shown engaged with the sleeping or resting structure 1122 in a lying position. In the schematic illustration, the actuation module 1132 is shown below the sleeping or resting structure 1122. The actuation module 1132 can be substantially analogous to the actuation module 208 of FIG. 2, redundant explanation of which is omitted herein for clarity. The actuation module 1132 can be associated with a sensing module 1136 and an ambient emission module 1140, as shown in FIG. 11C. The sensing module 1136 can be configured to detect one or more condition of a user. For example, the sensing module 1136 can be configured to detect an audible condition of the user 1110, such as snoring or a voice command. Additionally or alternatively, the sensing module 1136 can be configured to detect a force and/or motion input from the user, which can be indicative of a change in position of the user 1110 on the sleeping or resting structure 1122.

The actuation module 1132 can be configured to receive a signal from the sensing module 1136 and alter one or more characteristics of the sleeping or resting structure 1122. For example, the actuation module 1132 can be adapted to change a firmness of the sleeping or resting structure 1122 as a result of a user command. In other cases, the actuation module 1132 can be configured to change or reposition the user 1110, such as repositioning the user 1110 to provide the user 1110 with more support or firmness and/or reposition the user 1110 to mitigate sleeping apnea events, among other possibilities. Additionally or alternatively, the ambient emission module 1140 can be configured to receive a signal from the sensing module 1136 and alter one or more characteristics of the sleeping volume 1104. For example, the ambient emission module 1140 can be configured to introduce certain smells into the sleeping volume 1104 based on a detected condition of the user 1110. Further, the ambient emission module 1140 can be configured to introduce light, vibratory, heat, and other ambient effects 1106 into the sleeping volume 1104. In some cases, the actuation module 1132 and the ambient emission module 1140 can cooperate to produce combinative effects that can mitigate a detected condition. As one example, the sensing module 1136 can detect excessive movement of the user 1110 that is associated with restlessness. In turn, the adaptive actuation system 1130 can adjust a firmness of the sleeping or resting structure 1102 in connection with the ambient emission module 1140 introducing pleasing smells and calming sounds that can facilitate a restful sleep.

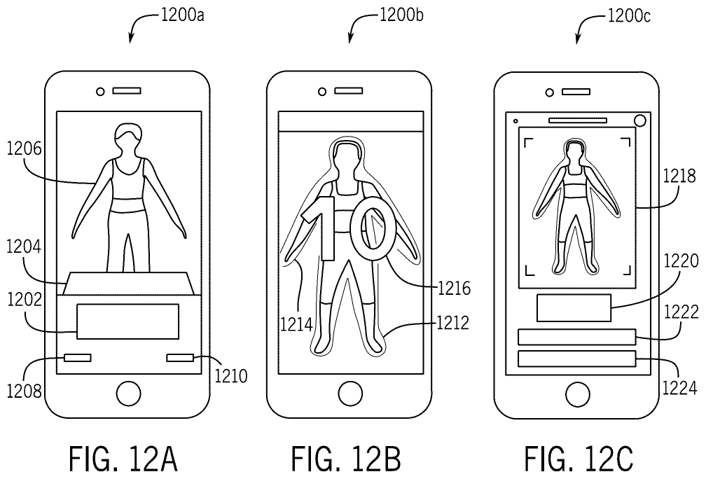

The sleeping system 1100 can be operated in part by mapping a body contour of the user 1110. The adaptive actuation system 1130 can in be configured to modify the sleeping or resting structure 1122 based on the contour of the body in order to deliver a customized sleeping experience. In some cases, the body mapping can be facilitated using a smartphone or other electronic device. For example and with reference to FIGS. 12A-12C, an electronic device is used to capture an image of the user 1110 in order to calibrate the sensors of the adaptive actuation system 1130. With reference to FIG. 12A, a first user interface 1200a is shown. The first interface 1200a can include information associated with initiating a calibration process. The first interface 1200a can include a prompt 1202 that includes a message indicating the proper placement of the electronic device or camera in order to capture an image of the user for calibration. The first interface 1200a can further include a table 1204 and an avatar 1206. The table 1204 can represent a reference object of the frame of the captured image. The avatar 1206 can convey information to the user regarding the appropriate position relative to the reference object for capturing the image. An exit button 1208 can be provided to stop the calibration sequence. A commence button 1210 can also be provided to begin a calibration process.

With reference to FIG. 12B, a second user interface 1200b is shown in which a calibration process is initiated. For example, the second interface 1200b presents a user image 1212 and an overlay 1214. The overlay 1214 can represent a targeted and/or an approximated body contour of the user, as presently captured in the user image 1212. A countdown 1216 is also provided which indicates the status of the calibration process. With reference to FIG. 12C, a third user interface 1200c is shown in which the calibration process is finalized. For example, the third interface 1200c presents a resulting match 1218 of the user image 1212 and the overlay 1214. A prompt 1220 can be provided, including information that requests user review of the resulting match 1218. At the third interface 1200c, a user can review the fit of the overlay 1214 with the user image 1212. If the resulting match 1218 is unacceptable, the user can select a retake button 1222 in order to revert to the second interface 1200b. In the event that the resulting match 1218 is acceptable, an acceptance button 1224 can be selected in order to complete the calibration process.

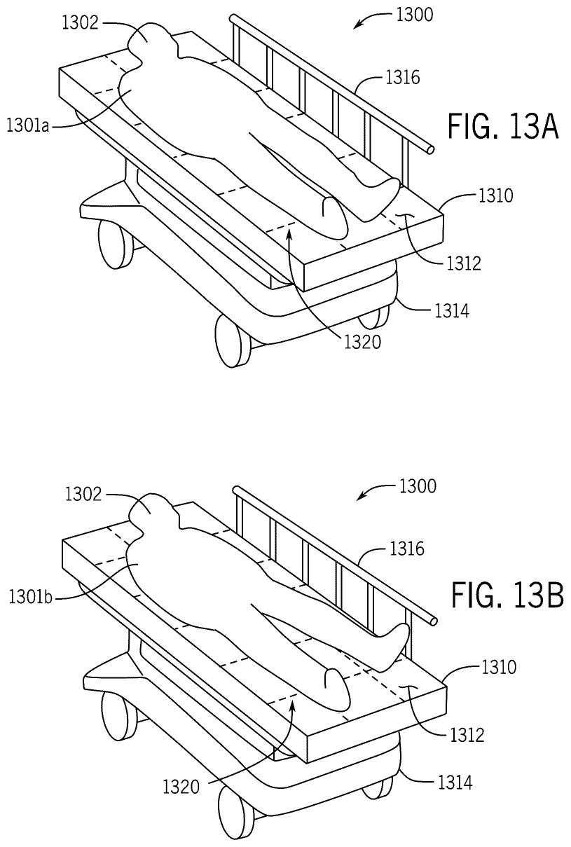

In another example, the actuation assemblies of the present disclosure can be implemented in a flexible fabric that forms a portion of a medical table. For example and as shown in FIG. 13A, a medical table 1300 is shown as having a flexible fabric 1310. The flexible fabric 1310 can define a medical surface 1312 that is configured to engage a user 1302 in a convalescing position. The medical table 1300 can be a hospital bed or other device that is used in the treatment and care of patients. In the example of FIG. 13A, the hospital bed 1300 is shown as including a wheel assembly 1314 and a rail 1316. In other examples, the hospital bed 1300 can include other components that facilitate the treatment of the user 1302.

The flexible fabric 1310 can be associated an actuation assembly 1320. The actuation assembly 1320 can be substantially analogous to the actuation assemblies and modules described herein, such as the actuation module 208 of FIG. 2; redundant explanation of which is omitted here for clarity. The actuation assembly 1320 can be configured to manipulate the flexible fabric 1310. For example, the actuation assembly 1320 can be configured to change characteristics of the medical surface 1312, such as firmness, or other characteristics in order to support the healing of the user 1302. In some cases, the manipulation of the flexible fabric 1310 can occur in response to a detection of a condition of the user 1302. The condition can include the position and/or movement of the user 1302. Additionally or alternatively, the condition can be associated with a medical diagnosis, and the actuation assembly 1320 can be configured to manipulate the user 1302 in order to facilitate a course of treatment. In some cases, the actuation assembly 1320 can be configured to manipulate the medical surface 1312 in a manner that causes movement of the user 1302. For example in FIG. 13A, the user 1302 is shown in a first position 1301a. The actuation assembly 1320 can operate to move the user 1302 into a second position 1301b, as shown in FIG. 13B. The movement of the user 1302 can occur substantially automatically and without direct input from medical personnel. This can be beneficial in order to support patient movement during treatment, in order to prompt circulation and decrease the prevalence of bed sores, and the like.

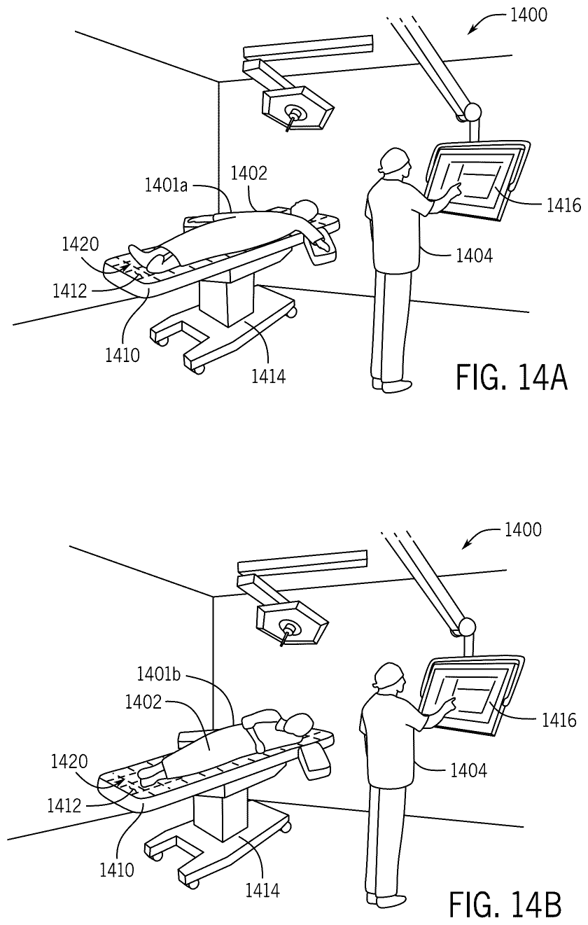

In another example, the actuation assemblies of the present disclosure can be implemented in a flexible fabric that forms a portion of a surgical table. For example and as shown in FIG. 14A, a surgical table 1400 is shown as having a flexible fabric 1410. The flexible fabric 1410 can define a surgical surface 1412 that is configured to engage a user 1402 in a surgical position. The surgical table 1400 can be associated with an operating room and/or other systems and components that facilitate the operation of surgical procedures. In the example of FIG. 14A, the surgical table 1400 is shown as including a wheel assembly 1414. In other examples, the surgical table 1400 can include other components that facilitate the surgical treatment of the user 1402. The surgical table 1400 is also shown as being associated with a control station 1416. The control station 1416 can be operatively associated with an actuation assembly 1420. A medical operator 1404 can use the control station 1416 to control the operation of the actuation assembly 1420.

The flexible fabric 1410 can be associated the actuation assembly 1420. The actuation assembly 1420 can be substantially analogous to the actuation assemblies and modules described herein, such as the actuation module 208 of FIG. 2; redundant explanation of which is omitted here for clarity. The actuation assembly 1420 can be configured to manipulate the flexible fabric 1410. For example, the actuation assembly 1420 can be configured to change characteristics of the surgical surface 1412, such as firmness, or other characteristics, in order to support the healing of the user 1402. In some cases, the manipulation of the flexible fabric 1410 can occur in response to a detection of a condition of the user 1402. The condition can include the position and/or movement of the user 1402. Additionally or alternatively, the condition can be associated with a medical diagnosis, and the actuation assembly 1420 can be configured to manipulate the user 1402 in order to facilitate a surgical operation. In some cases, the actuation assembly 1420 can be configured to manipulate the surgical surface 1412 in a manner that causes movement of the user 1402. For example in FIG. 14A, the user 1402 is shown in a first position 1401a. The actuation assembly 1420 can operate to move the user 1402 into a second position 1401b, as shown in FIG. 14B. The movement of the user 1402 can occur substantially automatically and without direct input from the medical personnel 1404. In other cases, the medical personnel 1404 can use the control station 1416 to adapt the operation of the actuation assembly 1420 to the user 1402 and real-time conditions of surgery. This can be beneficial in order to move and position the user 1402 in an appropriate manner during surgery.

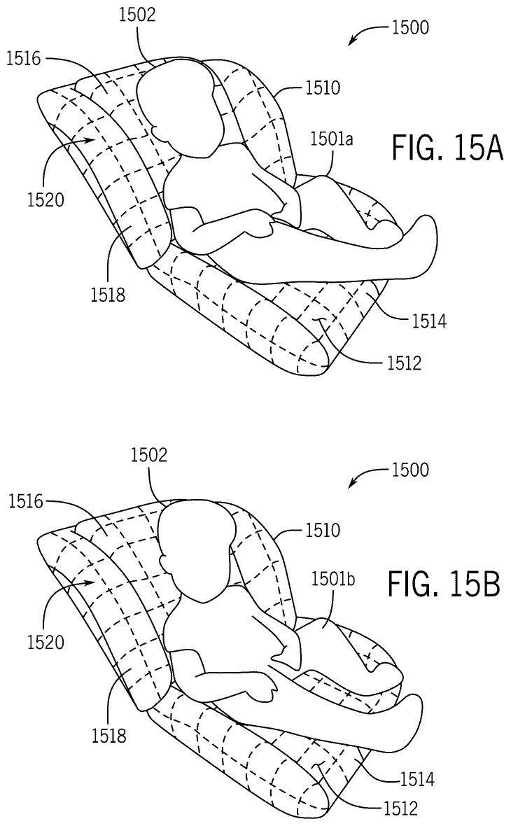

In another example, the actuation assemblies of the present disclosure can be implemented in a flexible fabric that forms a portion of a car seat. For example and as shown in FIG. 15A, a car seat 1500 is shown as having a flexible fabric 1510. The flexible fabric 1510 can define a sitting surface 1512 that is configured to engage a user 1502 in a sitting position. The car seat 1500 can be a device that is used to safely transport children in a motor vehicle. In the example of FIG. 15A, the car seat 1500 is shown as including a seat portion 1514, a back support portion 1516, and a side bumper portion 1518. The flexible fabric 1510 can extend over one or more of the seat portion 1514, the back support portion 1516, and the side bumper portion 1518. In other examples, the car seat 1500 can include other components that facilitate the transportation of the user 1502.

The flexible fabric 1510 can be associated an actuation assembly 1520. The actuation assembly 1520 can be substantially analogous to the actuation assemblies and modules described herein, such as the actuation module 208 of FIG. 2; redundant explanation of which is omitted here for clarity. The actuation assembly 1520 can be configured to manipulate the flexible fabric 1510. For example, the actuation assembly 1520 can be configured to change characteristics of the sitting surface 1512, such as firmness, or other characteristics in order to support the sitting of the user 1502. In some cases, the manipulation of the flexible fabric 1510 can occur in response to a detection of a condition of the user 1502. The condition can include the position and/or movement of the user 1502. In some cases, the actuation assembly 1520 can be configured to manipulate the sitting surface 1512 in a manner that causes movement of the user 1502. For example in FIG. 15A, the user 1502 is shown in a first position 1501a. The actuation assembly 1520 can operate to move the user 1502 into a second position 1501b, as shown in FIG. 15B. The movement of the user 1502 can occur substantially automatically and without direct input from another associated user, such as a parent of a child. This can be beneficial in order to support movement of the user 1502 during prolonged periods of sitting, in order to prompt circulation and enhance the usability of the car seat 1500 for prolonged durations.

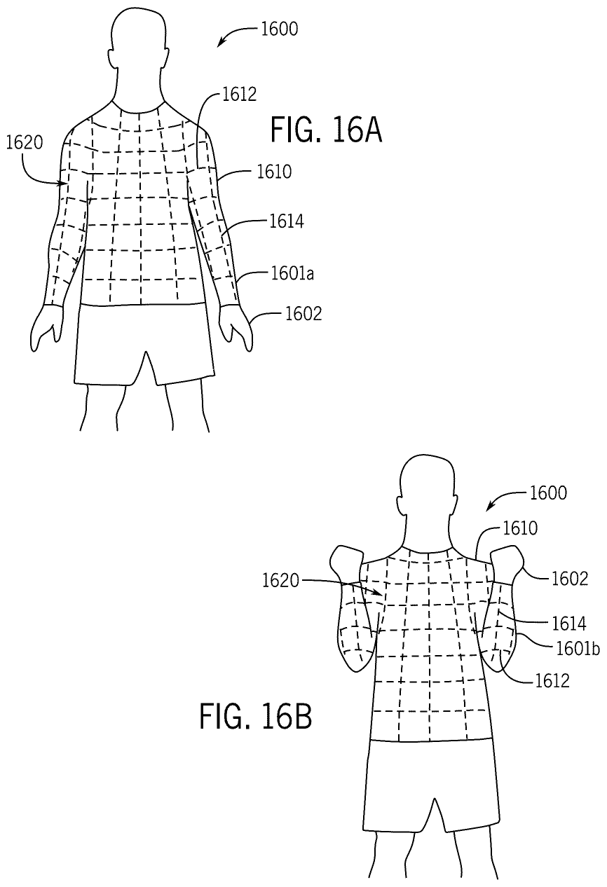

In another example, the actuation assemblies of the present disclosure can be implemented in a flexible fabric that forms a portion of a garment. For example and as shown in FIG. 16A, a garment 1600 is shown as having a flexible fabric 1610. The flexible fabric 1610 can define an interface that is configured to engage a user 1602 such that the garment 1600 can be worn by the user 1602. The garment 1600 can be a shirt or upper body covering that is used to facilitate exercise by selectively increasing the resistance of the flexible fabric 1610. In the example of FIG. 16A, the garment 1600 is shown as including bicep portion 1612 and a forearm portion 1614. In other examples, the garment 1600 can include other components that facilitate the exercise of the user 1602.

The flexible fabric 1610 can be associated an actuation assembly 1620. The actuation assembly 1620 can be substantially analogous to the actuation assemblies and modules described herein, such as the actuation module 208 of FIG. 2; redundant explanation of which is omitted here for clarity. The actuation assembly 1620 can be configured to manipulate the flexible fabric 1610. For example, the actuation assembly 1620 can be configured to change characteristics of one or both of the bicep portion 1612 and the forearm portion 1614, such as firmness or stiffness, or other characteristics in order to support an exercise regime of the user 1602. For example, the actuation assembly 1620 can operate in order to resist movements of the user 1602 at the bicep portion 1612 and/or the forearm portion 1614 in order to facilitate resistance training. In some cases, the manipulation of the flexible fabric 1610 can occur in response to a detection of a condition of the user 1602. The condition can include position and/or movement of the user 1602. For example in FIG. 16A, the user 1602 is shown in a first position 1601a. The actuation assembly 1620 can operate to increase resistance in the bicep portion 1612 and/or the forearm portion 1614 as the user 1602 moves between the first position 1601a and a second position 1601b, as shown in FIG. 16B. The change in resistance can occur substantially automatically and without direct input from the user 1602. This can be beneficial in order to support an exercise regime without the need for weights and other auxiliary equipment.

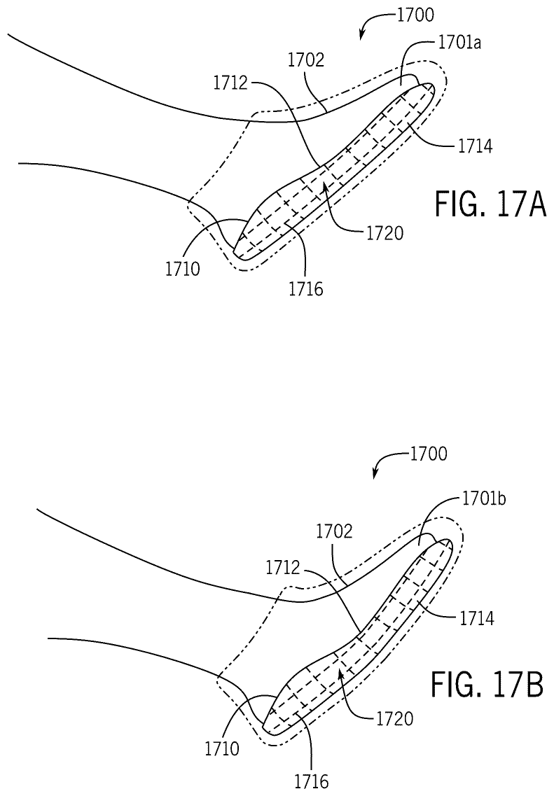

In another example, the actuation assemblies of the present disclosure can be implemented in a flexible fabric that forms a portion of an insole. For example and as shown in FIG. 17A, an insole 1700 is shown as having a flexible fabric 1710. The flexible fabric 1710 can define a stepping surface 1712 that is configured to engage a user 1702 in a standing position. The insole 1700 can be a device that is inserted into a shoe, cast, boot, or other component configured to be worn on a foot of the user 1702. In the example of FIG. 17A, the insole 1700 is shown as including a forward portion 1714 configured to engage a ball of the foot and a rear portion 1716 configured to engage an arch of the foot. In other examples, the insole 1700 can include other components that facilitate the stepping of the user 1702.

The flexible fabric 1710 can be associated an actuation assembly 1720. The actuation assembly 1720 can be substantially analogous to the actuation assemblies and modules described herein, such as the actuation module 208 of FIG. 2; redundant explanation of which is omitted here for clarity. The actuation assembly 1720 can be configured to manipulate the flexible fabric 1710. For example, the actuation assembly 1720 can be configured to change characteristics of the stepping surface 1712, such as firmness, or other characteristics in order to support the foot of the user 1702. In some cases, the manipulation of the flexible fabric 1710 can occur in response to a detection of a condition of the user 1702. The condition can include the position and/or movement of the user 1702. Additionally or alternatively, the condition can be associated with a medical diagnosis, and the actuation assembly 1720 can be configured to manipulate the user 1702 in order to facilitate a course of treatment, including mitigation of joint pain. In some cases, the actuation assembly 1720 can be configured to manipulate the stepping surface 1712 in a manner that causes movement of the user 1702. For example in FIG. 17A, the foot of the user 1702 is shown in a first position 1701a. The actuation assembly 1720 can operate to move the foot of the user 1702 into a second position 1701b, as shown in FIG. 17B. The movement of the user 1702 can occur substantially automatically and without direct input from the user 1702 during use, including walking or running. This can be beneficial in order to provide adaptive foot support during walking, running, exercise and/or other activities, responsive to the condition of the user 1702 and the environment.

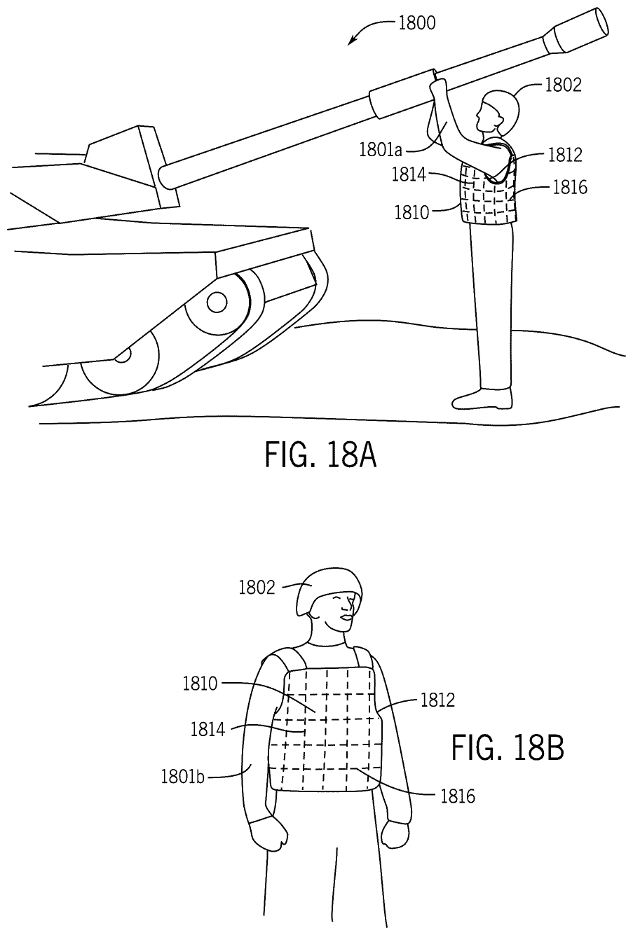

In another example, the actuation assemblies of the present disclosure can be implemented in a flexible fabric that forms a portion of a bullet-proof vest. For example and as shown in FIG. 18A, a bullet-proof vest 1800 is shown as having a flexible fabric 1810. The flexible fabric 1810 can define an interface 1812 that is configured to engage a user 1802 such that the bullet-proof vest 1800 can be worn by the user 1802. The bullet-proof vest 1800 can be an upper body covering that can be used to resist a bullet. The resistance of the flexible fabric 1810 can be modified to permit movement of the user 1802 in different combat scenarios. In the example of FIG. 18A, the bullet-proof vest 1800 is shown as including a first chest portion 1814 and a second chest portion 1816. In other examples, the bullet-proof vest 1800 can include other components that facilitate the protection of the user 1802.

The flexible fabric 1810 can be associated an actuation assembly 1820. The actuation assembly 1820 can be substantially analogous to the actuation assemblies and modules described herein, such as the actuation module 208 of FIG. 2; redundant explanation of which is omitted here for clarity. The actuation assembly 1820 can be configured to manipulate the flexible fabric 1810. For example, the actuation assembly 1820 can be configured to change characteristics of the interface 1812, such as firmness, or other characteristics in order to support the movement of the user 1802. In some cases, the manipulation of the flexible fabric 1810 can occur in response to a detection of a condition of the user 1802. The condition can include the position and/or movement of the user 1802. For example in FIG. 18A, the user 1802 is shown in a first position 1801a. The actuation assembly 1820 can operate to increase resistance in the first chest portion 1814 and/or the second chest portion 1816 as the user 1802 moves between the first position 1801a and a second position 1801b, as shown in FIG. 18B. The change in resistance of the flexible fabric 1810 can occur substantially automatically and without direct input from the user 1802. This can be beneficial in order to support a transition from a non-combat scenario, as shown in FIG. 18A, to a combat scenario, as shown in FIG. 18B.

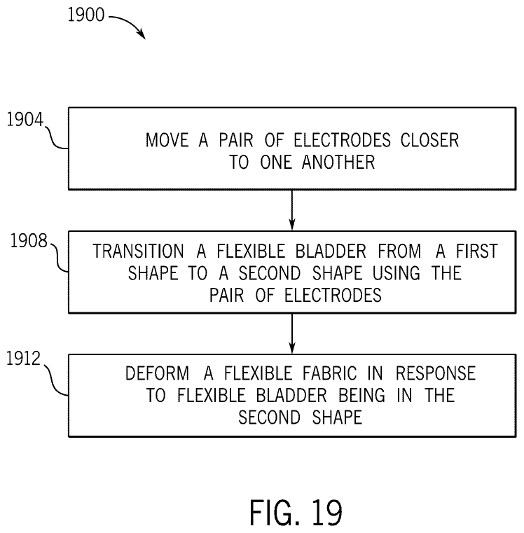

To facilitate the reader's understanding of the various functionalities of the embodiments discussed herein, reference is now made to the flow diagram in FIG. 19, which illustrates a process 1900. While specific steps (and orders of steps) of the methods presented herein have been illustrated and will be discussed, other methods (including more, fewer, or different steps than those illustrated) consistent with the teachings presented herein are also envisioned by and encompassed within the present disclosure.

At operation 1904, a pair of electrodes are moved closer to one another. For example and with reference to FIGS. 4A and 4B, the first electrode 424 and the second electrode 428 can be moved closer to one another. The second electrode 428 can be connected to the current supply 430. The first electrode 424 can be connected to the ground 426. An electrical charge can be applied to the first and second electrodes 424, 428. The electrical change can induce an electromagnetic field that draws the first and second electrodes 424, 428 closer to one another.

At operation 1908, a flexible bladder is transitioned from a first shape to a second shape using the first and second electrodes 424, 428. For example and with reference to FIGS. 4A and 4B, the movement of the first and second electrodes 424, 428 can displace the fluid 432 within the portion of the flexible bladder 436 arranged between the first and second electrodes 424, 428. The fluid 432 can be displaced toward opposite ends of the flexible bladder 436 and cause the flexible bladder 436 to stretch and expand. For example, the flexible bladder 436 can transition from having a first shape with the first height h₁ to a second shape with the second, greater height h₂.

At operation 1912, a flexible fabric is deformed in response to the flexible bladder 436 being in the second shape. For example and with reference to FIG. 4B, the transition of the flexible bladder 436 to the second shape can cause a deformation of the flexible fabric 400. The flexible bladder 436 can be arranged at least partially within a fabric region 408 of the flexible fabric 400. The fabric region 408 can extend toward the flexible outer surface 404. The flexible bladder 436 can expand at least partially within the fabric region 408 and cause the flexible outer surface 404 to deform and define the manipulated portion 410. The manipulated portion 410 can have a deformed height Δₛ. A value of the deformed height Δₛ can correspond to a value of the second height h₂ of the flexible bladder 436 in the second shape.

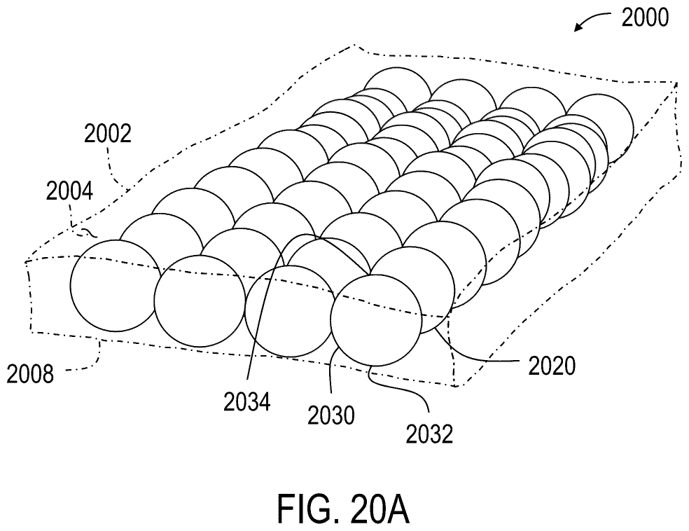

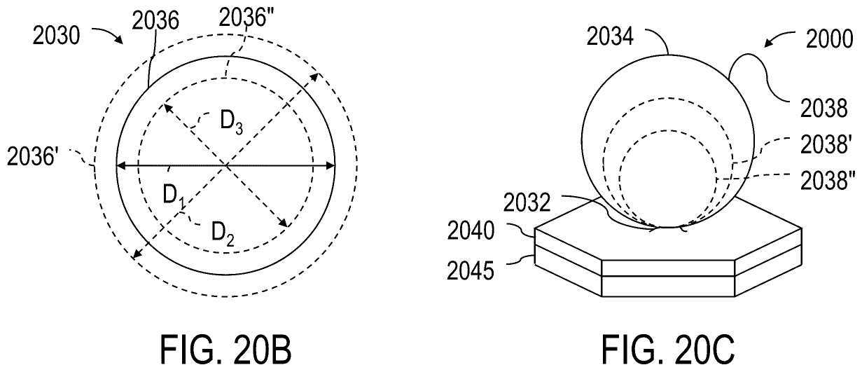

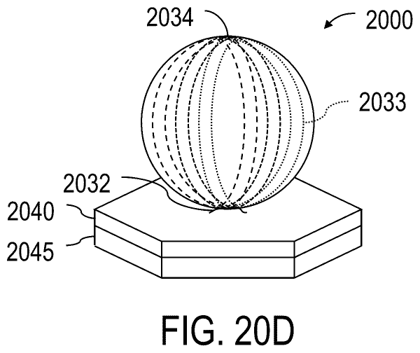

FIGS. 20A-20D illustrate an example flexible fabric 2000 including an actuation assembly 2020. The actuation assembly 2020 includes a collection of size-changing materials and components (e.g., size-changing components 2030) configured to manipulate the flexible fabric 2000. The flexible fabric 2000 is illustrated in FIG. 20A as including a fabric outer layer 2002, a flexible surface 2004 and a fabric region 2008. The actuation assembly 2020 and/or the size-changing components 2030 can be held at least partially within or otherwise interface with the fabric region 2008. The actuation assembly 2020 is operable to cause a deformation of the flexible outer surface 2004 upon actuation.

To facilitate the foregoing, the actuation assembly 2020 can include an array of size-changing components 2030. In the example of FIGS. 20A-20D, the size-changing components 2030 are sphere-shaped; however, the size-changing components 2030 can include other shapes, such as polyhedrons, cylinders, cones, or any other suitable shapes. The size-changing components 2030 can be formed of materials that change size (e.g., diameter) or other dimensions upon the receipt of heat, light, electrical stimuli, or other energy. For example, as illustrated in FIG. 20B, which shows a detailed view of a size-changing component 2030, a diameter of the size-changing component 2030 can grow from a first diameter D₁ in a first body arrangement 2036 to a second diameter D₂ in a second body arrangement 2036′ upon a first change in supplied energy. The diameter of the size-changing component 2030 can shrink from the first diameter D₁ in the first body arrangement 2036 to a third diameter D₃ in a third body arrangement 2036″ upon a second change in supplied energy opposite the first change. Varying the size or other dimensions of the size-changing components 2030 can cause deformations in the flexible fabric 2000, such as altering the firmness of the flexible fabric 2000.

In some examples, the size-changing components 2030 can be formed of materials having high volumetric coefficients of expansion. In examples in which the size-changing components 2030 change size in response to heat energy supplied to the size-changing components 2030, the size-changing components can include materials having volumetric coefficients of thermal expansion greater than about 20/° C. greater than about 30/° C. greater than about 50/° C., greater than about 100/° C., or the like. Growth and shrinkage of the size-changing components 2030 can be configured to cause a deformation of the flexible surface 2004. For example, application of energy to the size-changing components 2030 in portions of the flexible fabric 2000 can cause the size-changing components 2030 in those portions to grow, and can cause those portions to become more firm. Cessation of the application of energy to the size-changing components 2030 in portions of the flexible fabric 2000 can cause the size-changing components 2030 in those portions to shrink, and can cause those portions to become less firm. In some examples, application of energy and cessation of the application of energy can cause the size-changing components to shrink and grow, respectively.

Each of the size-changing components 2030 can have a first end 2032 that can be fixed or otherwise attached to a base. The size-changing components 2030 can have a second end 2034 that is a free end opposite the first end 2032. The size-changing components 2030 can be configured to transition between a first configuration and a second configuration upon application of energy. In the example of FIGS. 20A-20D, the transition of the size-changing components 2030 can cause the second end 2034 to move relative to the fabric region 2008. The movement of the second end 2034 relative to the fabric region 2008 can be configured to cause a deformation of the flexible surface 2004.

With reference to FIG. 20C, a size-changing component 2030 is shown associated with a base 2040 and an energy source 2045. The size-changing component 2030 can be connected to the base 2040 at the first end 2032. The energy source 2045 can be coupled with the base 2040 and/or the size-changing component 2030 and can be operable to deliver energy to the size-changing component 2030. For example, the size-changing component 2030 can be or include a size-changing component that changes diameter or the like when exposed to heat, light, electrical stimuli, or the like. The energy source 2045 can include a heat source, a light source, an electrical source, or the like that is configured to deliver energy to the size-changing component 2030 and cause the size-changing component 2030 to change size.

In the configuration of FIG. 20C, the size-changing component 2030 is shown as having a first body arrangement 2038. For example, in the first body arrangement 2038, the size-changing component 2030 can substantially extend and protrude from the base 2040. The first body arrangement 2038 can correspond to a state of the actuation assembly 2020 in which the size-changing component 2030 receives energy from the energy source 2045. FIG. 20C also shows a second body arrangement 2038′ and a third body arrangement 2038″. The second body arrangement 2038′ and the third body arrangement 2038″ can correspond to a configuration of the size-changing component 2030 in which the size-changing component 2030 receives less or no energy from the energy source 2045. In this regard, the size-changing component 2030 can be configured to transition between a range of configurations and sizes between the first body arrangement 2038 and the third body arrangement 2038″ based on a quantity of energy received from the energy source 2045. In some examples, the size-changing component 2030 can operate in an opposite manner, such that the third body arrangement 2038″ corresponds to a configuration in which the size-changing component 2030 receives energy from the energy source 2045 and the second body arrangement 2038′ and the first body arrangement 2038 correspond to configurations in which the size-changing component 2030 receives less or no energy from the energy source 2045.

The introduction energy heat to the size-changing component 2030 can cause the size-changing component 2030 to exhibit an increased diameter Δd. The increased diameter Δd can be at least 110% of an initial diameter di, at least 130% of the initial diameter di, at least 150% of the initial diameter di, or the like. The value of the increased diameter Δd can be partially based on the amount of energy added to the size-changing component 2030. For example, in a first instance, a first quantity of energy can be introduced to the size-changing component 2030 by the energy source 2045 in order to increase the diameter of the size-changing component 2030 by a first amount. In a second instance, a second, greater quantity of energy can be introduced to the size-changing component 2030 by the energy source 2045 in order to increase the diameter of the size-changing component 2030 by a second, greater amount.

In some examples, the size-changing components 2030 can be formed from any of the shapeable materials discussed above, such as the shapeable materials discussed in reference to FIGS. 6-9C. For example, as illustrated in FIG. 20D, a size-changing component 2030 can include a plurality of shapeable materials 2033, which are arranged to form the size-changing component 2030. Altering the shape of the shapeable materials 2033, such as by changing energy supplied to the shapeable materials 2033, can alter the tension in the size-changing component 2030, and can alter the size of the size-changing component 2030. More specifically, changing the energy supplied to the shapeable materials 2033 can change the shape of the shapeable materials 2033 from a first shape to a second shape, which changes the size of the size-changing component 2030 from a first size to a second size.

In some examples, the size-changing components 2030 of the actuation assembly 2020 can be replaced by a phase change polymer or other phase change material (collectively referred to as phase change materials or PCMs). The phase change materials can be manipulatable by receipt of heat energy. Joule heating or the like can be used to heat the phase change materials to change a phase of the phase change materials. In some examples, the phase change materials can transition from a solid state to a liquid state upon the receipt of heat, and can transition back from the liquid state to the solid state upon cessation of the receipt of heat. Thus, application of heat energy to the phase change materials in portions of the flexible fabric 2000 can cause those portions to soften or become less firm. Removal of heat energy from the phase change materials in portions of the flexible fabric 2000 can cause those portions to harden or become more firm.

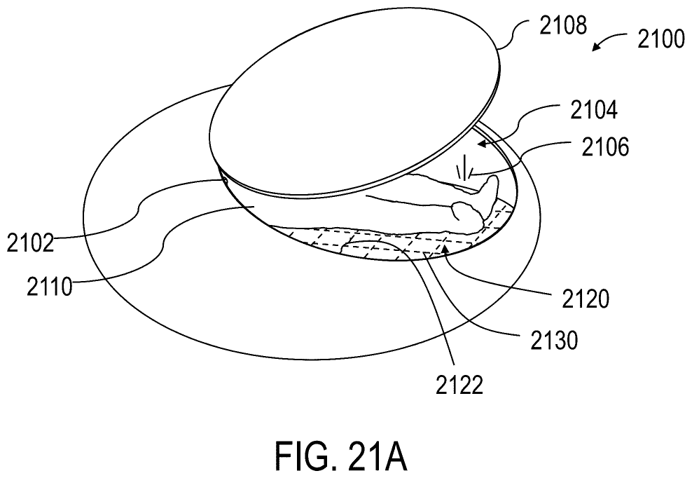

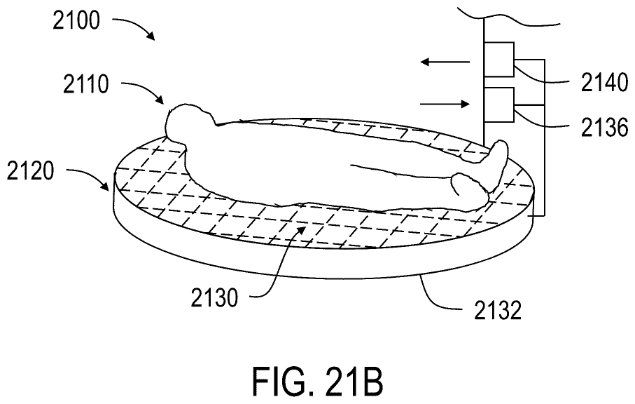

FIGS. 21A and 21B illustrate an example of a sealable sleeping system 2100. The sealable sleeping system 2100 can implement an adaptive actuation system, such as those described above, to control one or more characteristics of a mattress based on a user condition. The sealable sleeping system 2100 can be adapted to contribute to an immersive sleeping experience. For example, the sealable sleeping system 2100 can include a pod 2102 that defines a sleeping volume 2104. The sleeping volume 2104 can provide ambient effects 2106, such as lights, smells, sounds, and the like. The pod 2102 can include a door 2108 or other sealable hatch, and the ambient effects 2106 can further include specific gas concentrations, pressures, therapeutic gases (e.g., hallucinogenics, other drug or medical treatments, or the like), and the like. The sleeping volume 2104 can be configured to hold a mattress 2120 and a user 2110 therein. The pod 2102 can be substantially spherical, egg-shaped, cylindrical, rounded, rectangular, or any other suitable form in shape. The pod 2102 can form a sealable enclosure around the user 2110. The mattress 2120 can include a sleeping surface 2122 that is configured to engage the user 2110 in the lying position within the sleeping volume 2104. The mattress 2120 can include an adaptive actuation system 2130, such as any of the adaptive actuation systems described herein. The adaptive actuation system 2130 is shown in the example of FIGS. 21A and 21B with a representative grid (shown in phantom). The adaptive actuation system 2130 can be configured to modify the sleeping surface 2122, as described herein.

The sealable sleeping system 2100 can be configured to detect a condition of the user 2110 and alter one or more characteristics of a sleeping experience in response to the condition of the user 2110. FIG. 21B illustrates a schematic diagram of the sealable sleeping system 2100. In the schematic diagram of FIG. 21B, the user 2110 is shown engaged with the sleeping surface 2122 in a lying position. In the schematic illustration, an actuation module 2132 is shown below the sleeping surface 2122. The actuation module 2132 can be substantially analogous to the actuation module 208 of FIG. 2 and/or the actuation module 1132 of FIG. 11C, redundant explanation of which is omitted herein for clarity. The actuation module 2132 can be associated with a sensing module 2136 and an ambient emission module 2140, as shown in FIG. 21B. The sensing module 2136 can be configured to detect one or more conditions of the user 2110. For example, the sensing module 2136 can be configured to detect an audible condition of the user 2110, such as snoring or a voice command. The sensing module 2136 can be configured to detect a force and/or motion input from the user 2110, which can be indicative of a change in position of the user 2110 on the sleeping surface 2122.

In some examples, the sensing module 2136 can interface with one or more external devices in order to obtain additional information indicative of conditions of the user 2110. For example, the sensing module 2136 can interface with a user device, such as a smartphone, other electronic device, or the like to collect and obtain data relevant to conditions of the user 2110 throughout the day. The data may include daily activities, oxygen levels, heat rate, diet, consumables, environmental factors, and any other data relevant to conditions of the user 2110. The sensing module 2136 can interface with weather services, and the like to obtain local environmental data, such as concentrations of pollutants and the like in the environment. The sensing module 2136 can include sensors to detect brainwaves and the like. The data collected by the sensing module 2136 can be used to customize the sleep experience provided to the user 2110.

The actuation module 2132 can be configured to receive a signal from the sensing module 2136 and alter one or more characteristics of the sleeping surface 2122. For example, the actuation module 2132 can be adapted to change a firmness of the sleeping surface 2122 as a result of a user command. In some examples, the actuation module 2132 can be configured to change or reposition the user 2110, such as repositioning the user 2110 to provide the user 2110 with more support or firmness and/or reposition the user 2110 to mitigate sleeping apnea events, among other possibilities.

Additionally or alternatively, the ambient emission module 2140 can be configured to receive a signal from the sensing module 2136 and to alter one or more characteristics of the sleeping volume 2104. For example, the ambient emission module 2140 can be configured to introduce certain smells into the sleeping volume 2104, to alter the pressure of the sleeping volume 2104, to alter gaseous concentrations in the sleeping volume 2104, to administer drugs or other therapies into the sleeping volume 2104, or the like based on a detected condition of the user 2110, either currently, or based on data relevant to daily activities of the user 2110. In some examples, the sealable sleeping system 2100 can be a pressure chamber configured to perform hyperbaric oxygen therapy or the like. In some examples, the sealable sleeping system 2100 can be configured to provide drugs or other therapies to the user 2110 during sleep, such as micro-dosing hallucinogenics to aid in focus, help with ADHD symptoms, or the like. In some examples, the ambient emission module can manipulate the sleeping or resting environment of the pod 2102 to optimize the brainwaves of the user 2110 during sleep. Further, the ambient emission module 2140 can be configured to introduce light, vibratory, heat, and other ambient effects 2106 into the sleeping volume 2104. In some cases, the actuation module 2132 and the ambient emission module 2140 can cooperate to produce combinative effects that can mitigate a detected condition. As one example, the sensing module 2136 can detect excessive movement of the user 2110 that is associated with restlessness. The adaptive actuation system 2130 can adjust a firmness of the sleeping surface 2102 in connection with the ambient emission module 2140 introducing pleasing smells and calming sounds that can facilitate a restful sleep.

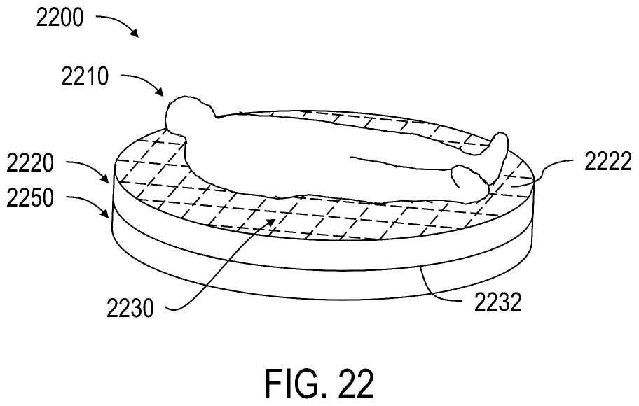

FIG. 22 illustrates an example of a sleeping system 2200. The sleeping system 2200 can implement an adaptive actuation system, such as those described above, to control one or more characteristics of a mattress based on a user condition. The sleeping system 2200 can be adapted to contribute to an immersive sleeping experience. For example, the sleeping system 2200 can include a temperature control system 2250 and a mattress 2220 on the temperature control system 2250. The mattress 2220 can be configured to hold a user 2210 thereon. The mattress 2220 can include a sleeping or resting structure 2222 that is configured to engage the user 2210 in a sitting, prone, or semi-prone position. The mattress 2220 can include an adaptive actuation system 2230, such as any of the adaptive actuation systems described herein. The adaptive actuation system 2230 is shown in the example of FIG. 22 with a representative grid (shown in phantom). The adaptive actuation system 2230 can be configured to modify the sleeping or resting structure 2222, as described herein.

In the example of FIG. 22, an actuation module 2232 is shown below the sleeping or resting structure 2222. The actuation module 2232 can be substantially analogous to the actuation module 208 of FIG. 2 and/or the actuation module 1132 of FIG. 11C, redundant explanation of which is omitted herein for clarity. The actuation module 2232 can be configured to receive signals from a sensing module or the like and alter one or more characteristics of the sleeping or resting structure 2222. For example, the actuation module 2232 can be adapted to change a firmness of the sleeping or resting structure 2222 as a result of a user command. In some examples, the actuation module 2232 can be configured to change or reposition the user 2210, such as repositioning the user 2210 to provide the user 2210 with more support or firmness and/or reposition the user 2210 to mitigate sleeping apnea events, among other possibilities.

The temperature control system 2250 is illustrated as being disposed opposite the user 2210 relative to the mattress 2220 and the adaptive actuation system 2230. In some examples, the temperature control system 2250 can be between the mattress 2220 and the user 2210, between the adaptive actuation system 2230 and the mattress 2220, or the like. The temperature control system 2250 can include a fluid, and thermal energy can be supplied to the fluid to provide temperature control to the user 2210 through the sleeping system 2200. In some examples, the adaptive actuation system 2230 can include one or more air bladders, which can provide pressure and/or firmness control to the sleeping or resting structure 2222.

Other examples and implementations are within the scope and spirit of the disclosure and appended claims. For example, features implementing functions can also be physically located at various positions, including being distributed such that portions of functions are implemented at different physical locations. Also, as used herein, including in the claims, “or” as used in a list of items prefaced by “at least one of” indicates a disjunctive list such that, for example, a list of “at least one of A, B, or C” means A or B or C or AB or AC or BC or ABC (i.e., A and Band C). Further, the term “exemplary” does not mean that the described example is preferred or better than other examples.

The foregoing description, for purposes of explanation, uses specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A resting or sleeping system comprising:
a sleeping or resting structure configured to engage a user arranged in a sitting, prone or semi-prone position;
an actuation assembly configured to manipulate the sleeping or resting structure, wherein the sleeping or resting structure is responsive to energy provided by the actuation assembly; and
a chamber configured to manipulate a sleeping or resting environment of the user via control of at least one of a vibration, a light, a sound, or a gas.

2. The resting or sleeping system of claim 1, further comprising a sensing module configured to detect brainwaves of the user, wherein the chamber is configured to manipulate the sleeping or resting environment in response to the brainwaves detected by the sensing module.

3. The resting or sleeping system of claim 1, wherein the sleeping chamber is further configured to provide gaseous medical treatments to the user.

4. The resting or sleeping system of claim 1, wherein the actuation assembly comprises a size-changing component, wherein the size-changing component is configured to change size in response to energy supplied to the size-changing component, and wherein the sleeping or resting structure is responsive to the size of the size-changing component.

5. The resting or sleeping system of claim 4, wherein the size-changing component comprises a material having a volumetric coefficient of thermal expansion greater than 20/° C.

6. The resting or sleeping system of claim 4, wherein the size-changing component is spherical.

7. The resting or sleeping system of claim 4, wherein:
the size-changing component has a free end;
the free end is in a first position when the size-changing component has a first size; and
the free end is in a second position when the size-changing component has a second size.

8. A method for providing an immersive sleep experience comprising:
providing a flexible fabric configured to support a user in a sleeping volume;
causing a deformation of the flexible fabric by manipulating an actuator; and
altering at least one of a composition of gas or a pressure of gas in the sleeping volume.

9. The method of claim 8, wherein causing the deformation of the flexible fabric comprises transitioning a size-changing component from a first size to a second size by altering energy supplied to the size-changing component.

10. The method of claim 9, further comprising detecting a user input, wherein the user input comprises data from a user's smart device, wherein transitioning the size-changing component occurs in response to the detection of the user input.

11. The method of claim 10, wherein the data comprises one or more of daily activities, oxygen levels, heart rate, diet, consumables, or environmental factors.

12. The method of claim 8, further comprising detecting a user input, wherein the user input comprises data from a user's smart device, wherein altering at least one of the composition of gas or the pressure of gas in the sleeping volume occurs in response to the detection of the user input.

13. The method of claim 8, further comprising detecting the user's brainwaves, wherein altering at least one of the composition of gas or the pressure of gas in the sleeping volume occurs in response to the detection of the user's brainwaves.

14. The method of claim 8, further comprising altering thermal energy supplied to a fluid in a temperature control system to alter a temperature of the flexible fabric.

15. The method of claim 8, further comprising supplying a gaseous medical treatment to the sleeping volume.

16. A mattress comprising:
a sleeping or resting structure configured to engage a user arranged in a prone, semi-prone, or sitting position; and
an actuation assembly configured to manipulate the sleeping or resting structure, wherein the actuation assembly comprises a size-changing component configured to transition between a first size and a second size in response to the size-changing component receiving energy from an energy source, and wherein the sleeping or resting structure is responsive to the size-changing component having the first size or the second size.

17. The mattress of claim 16, wherein the size-changing component comprises a material having a volumetric coefficient of thermal expansion greater than 20/° C.

18. The mattress of claim 16, wherein:
the energy source comprises a heat source;
the heat source is configured to emit heat directed toward the size-changing component; and
the size-changing component is configured to transition from the first size to the second size upon the receipt of heat from the heat source.

19. The mattress of claim 16, wherein the actuation assembly comprises a plurality of size-changing components comprising the size-changing components, wherein the size-changing components are spherical.

20. The mattress of claim 16, wherein the size-changing component comprises a plurality of shapeable materials, wherein the shapeable materials are configured to transition from a first shape to a second shape upon the receipt of energy to transition the size-changing component from the first size to the second size.
